(19) United States
(12) Patent Application Publication
(10) Pub. No.: US 2014/0051803 A1
(43) Pub. Date: Feb. 20, 2014

(54) HIGH PURITY BISPHENOL-A AND POLYCARBONATE MATERIALS PREPARED THEREFROM

(71) Applicants: Johannes De Brouwer, Oisterwijk (NL); Paulus Johannes Maria Eijsbouts, Nieuwkijk (NL)

(72) Inventors: Johannes De Brouwer, Oisterwijk (NL); Paulus Johannes Maria Eijsbouts, Nieuwkijk (NL)

(73) Assignee: SABIC INNOVATIVE PLASTICS IP B.V., Bergen Op Zoom (NL)

(21) Appl. No.: 13/806,046

(22) PCT Filed: May 2, 2012
(86) PCT No.: PCT/IB2012/052199

§ 371 Date: Mar. 11, 2013

Related U.S. Application Data
(63) Continuation-in-part of application No. 13/099,026, filed on May 2, 2011.
Application No. 13/099,026 is a continuation-in-part of application No. 13/099,032, filed on May 2, 2011.
(60) Provisional application No. 61/618,360, filed on Mar. 30, 2012.

(51) Int. Cl. C08G 64/06; C08L 69/00; C07C 39/16

(52) U.S. Cl. CPC C08G 64/06; C07C 39/16; C08L 69/00
USPC 524/611; 568/723; 528/196; 525/462

(57) ABSTRACT

A modified ion exchange resin catalyst having an attached dimethyl thiazolidine promoter is disclosed. Also disclosed is a process for catalyzing condensation reactions between phenols and ketones, wherein reactants are contacted with a modified ion exchange resin catalyst having an attached dimethyl thiazolidine promoter. Also disclosed is a process for catalyzing condensation reactions between phenols and ketones that does not utilize a bulk promoter.

### TECHNICAL FIELD

The present disclosure relates to catalyst systems, and specifically to promoter ion exchange resin catalyst systems and the products derived from them.

### TECHNICAL BACKGROUND

Many conventional condensation reactions utilize inorganic acid catalysts, such as sulfuric acid or hydrochloric acid. Use of such inorganic acid catalysts can result in the formation of undesirable byproducts that must be separated from the reaction stream. Ion exchange resin catalyst systems can also be used, but the inherent low acid concentration can require the use of a promoter or rate accelerator.

When used as part of the catalyst system, reaction promoters can improve reaction rate and selectivity. In the case of the condensation of phenol and ketone to form bisphenol-A (BPA), reaction promoters can improve selectivity for the desired para-para BPA isomer.

Reaction promoters can be used as bulk promoters, where the promoter is present as an unattached molecule in the reaction medium, or as an attached promoter, where the promoter is attached to portion of the catalyst system.

In the synthesis of BPA, the use of 3-mercaptopropionic acid (3-MPA) as a promoter can produce a significant quantity of the less desirable o,p-BPA isomer, as opposed to the preferred p,p-BPA isomer.

Existing attached promoter systems, such as, pyridyl ethylmercaptons (PEM), can be susceptible to reactant impurities. For example, in the production of BPA, hydroxyacetone (HA) and methanol can be present in phenol and acetone reactants, respectively. As impurities, such as HA and methanol, can deactivate promoter systems, additional process steps to remove the impurities can be required. Such attached promoter systems can also be susceptible to impurities in recycle feeds of reaction processes, reducing the lifetime and performance of the catalyst system.

While much effort has been applied to the development and use of bulk and attached promoter systems, a need still exists for a manufacturing process and promoter catalyst system that can provide improved reaction rates, improved selectivity, and exhibit an improved tolerance for impurities over conventional systems. Thus, there is a need to address these and other shortcomings associated with existing promoter catalyst systems. These needs and other needs are satisfied by the compositions and methods of the present disclosure.

### SUMMARY

In accordance with the purpose(s) of the invention, as embodied and broadly described herein, this disclosure, in one aspect, relates to catalyst systems, and specifically to promoter ion exchange resin catalyst systems.

In one aspect, the present disclosure provides a catalyst system comprising a cross-linked, sulfonated ion exchange resin catalyst and a dimethyl thiazolidine promoter.

In another aspect, the present disclosure provides a catalyst system comprising a cross-linked, sulfonated ion exchange resin catalyst and a dimethyl thiazolidine promoter, wherein the cross-linked, sulfonated ion exchange resin comprises a plurality of sulfonic acid groups and has a degree of cross-linking of from about 1% to about 4%.

In another aspect, the present disclosure provides a catalyst system comprising a cross-linked, sulfonated ion exchange resin catalyst and a dimethyl thiazolidine promoter, wherein the dimethyl thiazolidine promoter is at least partially bound to the cross-linked, sulfonated ion exchange resin.

In another aspect, the present disclosure provides a catalyst system comprising a cross-linked, sulfonated ion exchange resin catalyst and a dimethyl thiazolidine promoter, wherein the dimethyl thiazolidine promoter is bound to from about 18% to about 25% of the sulfonic acid groups of the cross-linked, sulfonated ion exchange resin.

In another aspect, the present disclosure provides an attached promoter catalyst system comprising an ion exchange resin and a dimethyl thiazolidine promoter, wherein the catalyst system is more resistant to hydroxyacetone than a conventional bulk promoter system.

In another aspect, the present disclosure provides a method for catalyzing a condensation reaction, the method comprising contacting two or more reactants with a modified ion exchange resin catalyst in the absence of a bulk promoter.

In another aspect, the present disclosure provides a method for catalyzing a condensation reaction, the method comprising contacting two or more reactants with a modified ion exchange resin catalyst in the absence of a bulk promoter, wherein the modified ion exchange resin catalyst comprises a cross-linked, sulfonated ion exchange resin.

In another aspect, the present disclosure provides a method for catalyzing a condensation reaction, the method comprising contacting two or more reactants with a modified ion exchange resin catalyst in the absence of a bulk promoter, wherein the modified ion exchange resin catalyst comprises an attached dimethyl thiazolidine promoter.

In another aspect, the present disclosure provides a method for the production of bisphenol-A, the method comprising contact a phenol and at least one of a ketone, an aldehyde, or a combination thereof in the presence of an attached ion exchange resin catalyst comprising a dimethyl thiazolidine promoter, wherein the method does not comprise a pretreatment and/or purification step for the phenol, ketone, and/or aldehyde.

### BRIEF DESCRIPTION OF THE FIGURES

The accompanying figures, which are incorporated in and constitute a part of this specification, illustrate several aspects and together with the description serve to explain the principles of the invention.

FIG. 1 illustrates a comparison of p,p-BPA formation using an inventive catalyst, both with and without hydroxyacetone present.

FIG. 2 represents data from a methanol spiking experiment with the inventive catalyst system, illustrating the formation of p,p-BPA over time in the presence of methanol.

FIG. 3 represents data from a methanol spiking experiment with the inventive catalyst system, illustrating catalyst selectivity over time in the presence of methanol.

FIG. 4 represents data from a methanol spiking experiment with the inventive catalyst system, illustrating catalyst selectivity vs. methanol concentration.

FIG. 5 represents data from a methanol spiking experiment with the inventive catalyst system, illustrating p,p-BPA formation in the presence of varying methanol concentration.

FIG. 6 illustrates the yellowness index in a plastic 2.5mm color chip directly after molding as a function of monomer synthesis catalyst & promotor system.

FIG. 7 illustrates the yellowness index in a plastic 2.5mm color chip after 2,000 hrs of heat aging at 130° C. as a function of monomer synthesis catalyst & promotor system.

FIG. 8 illustrates the yellowness index in a plastic 2.5mm color chip directly after molding as a function of monomer organic purity and monomer synthesis catalyst & promotor system.

FIG. 9 illustrates the yellowness index in a plastic 2.5mm color chip after 2,000 hrs of heat aging at 130° C. as a function of monomer organic purity and monomer synthesis catalyst & promotor system.

Additional aspects of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such can, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, example methods and materials are now described.

As used in the specification and the appended claims, the singular forms “a,”“an” and “the” include plural referents unless the context clearly dictates otherwise. Thus, for example, reference to “a ketone” includes mixtures of two or more ketones.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint, and are independently combinable with endpoints of other expressed ranges for the same property. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or can not occur, and that the description includes instances where said event or circumstance occurs and instances where it does not. For example, the phrase “optionally substituted alkyl” means that the alkyl group can or can not be substituted and that the description includes both substituted and unsubstituted alkyl groups.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

References in the specification and concluding claims to parts by weight of a particular element or component in a composition or article denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “alkoxy” as used herein is an alkyl group bound through a single, terminal ether linkage; that is, an “alkoxy” group may be defined as —OR where R is alkyl as defined above. A “lower alkoxy” group is an alkoxy group containing from one to six carbon atoms.

The term “alkenyl group” as used herein is a hydrocarbon group of from 2 to 24 carbon atoms and structural formula containing at least one carbon-carbon double bond. Asymmetric structures such as (AB)C═C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aralkyl” as used herein is an aryl group having an alkyl, alkynyl, or alkenyl group as defined above attached to the aromatic group. An example of an aralkyl group is a benzyl group.

The term “hydroxyalkyl group” as used herein is an alkyl, alkenyl, alkynyl, aryl, aralkyl, cycloalkyl, halogenated alkyl, or heterocycloalkyl group described above that has at least one hydrogen atom substituted with a hydroxyl group.

The term “alkoxyalkyl group” is defined as an alkyl, alkenyl, alkynyl, aryl, aralkyl, cycloalkyl, halogenated alkyl, or heterocycloalkyl group described above that has at least one hydrogen atom substituted with an alkoxy group described above.

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

The term “carboxylic acid” as used herein is represented by the formula —C(O)OH.

The term “aldehyde” as used herein is represented by the formula —C(O)H.

The term “keto group” as used herein is represented by the formula —C(O)R, where R is an alkyl, alkenyl, alkynyl, aryl, aralkyl, cycloalkyl, halogenated alkyl, or heterocycloalkyl group described above.

The term “carbonyl group” as used herein is represented by the formula C═O.

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfo-oxo group” as used herein is represented by the formulas —S(O)₂R, —OS(O)₂R, or, —OS(O)₂OR, where R can be hydrogen, an alkyl, alkenyl, alkynyl, aryl, aralkyl, cycloalkyl, halogenated alkyl, or heterocycloalkyl group described above.

As used herein, unless specifically stated to the contrary, the term “polycarbonate” is intended to refer to compositions having repeating structural carbonate units of formula (1)

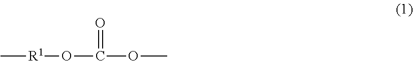

in which at least 60 percent of the total number of R¹ groups contain aromatic moieties and the balance thereof are aliphatic, alicyclic, or aromatic. In an aspect, each R¹ is a C₆₋₃₀ aromatic group, that is, contains at least one aromatic moiety. R¹ can be derived from a dihydroxy compound of the formula HO—R¹—OH, in particular of formula (2)

HO-A¹-Y¹-A²-OH (2)

wherein each of A¹ and A² is a monocyclic divalent aromatic group and Y¹ is a single bond or a bridging group having one or more atoms that separate A¹ from A². In an aspect, one atom separates A¹ from A². Specifically, each R¹ can be derived from a dihydroxy aromatic compound of formula (3)

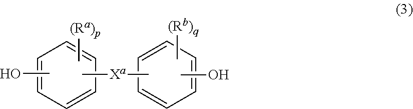

wherein R^{a} and R^{b} are each independently a halogen, C₁₋₁₂ alkoxy, or C₁₋₁₂ alkyl; and p and q are each independently integers of 0 to 4. It will be understood that R^{a} is hydrogen when p is 0, and likewise R^{b} is hydrogen when q is 0. Also in formula (3), X^{a} is a bridging group connecting the two hydroxy-substituted aromatic groups, where the bridging group and the hydroxy substituent of each C₆ arylene group are disposed ortho, meta, or para (specifically para) to each other on the C₆ arylene group. In an aspect, the bridging group X^{a} is single bond, —O—, —S—, —S(O)—, —S(O)₂—, —C(O)—, or a C₁₋₁₈ organic group. The C₁₋₁₈ organic bridging group can be cyclic or acyclic, aromatic or non-aromatic, and can further comprise heteroatoms such as halogens, oxygen, nitrogen, sulfur, silicon, or phosphorous. The C₁₋₁₈ organic group can be disposed such that the C₆ arylene groups connected thereto are each connected to a common alkylidene carbon or to different carbons of the C₁₋₁₈ organic bridging group. In one aspect, p and q is each 1, and R^{a} and R^{b} are each a C₁₋₃ alkyl group, specifically methyl, disposed meta to the hydroxy group on each arylene group.

In another aspect, X^{a} is a substituted or unsubstituted C₃₋₁₈ cycloalkylidene, a C₁₋₂₅ alkylidene of formula —C(R^{c})(R^{d})— wherein R^{c} and R^{d} are each independently hydrogen, C₁₋₁₂ alkyl, C₁₋₁₂ cycloalkyl, C₇₋₁₂ arylalkyl, C₁₋₁₂ heteroalkyl, or cyclic C₇₋₁₂ heteroarylalkyl, or a group of the formula —C(═R^{e})—wherein R^{e} is a divalent C₁₋₁₂ hydrocarbon group. groups of this type include methylene, cyclohexylmethylene, ethylidene, neopentylidene, and isopropylidene, as well as 2-[2.2.1]-bicycloheptylidene, cyclohexylidene, cyclopentylidene, cyclododecylidene, and adamantylidene.

In another aspect, X^{a} is a C₁₋₁₈ alkylene group, a C₃₋₁₈ cycloalkylene group, a fused C₆₋₁₈ cycloalkylene group, or a group of the formula —B¹-G-B²— wherein B¹ and B² are the same or different C₁₋₆ alkylene group and G is a C₃₋₁₂ cycloalkylidene group or a C₆₋₁₆ arylene group. For example, X^{a} can be a substituted C₃₋₁₈ cycloalkylidene of formula (4)

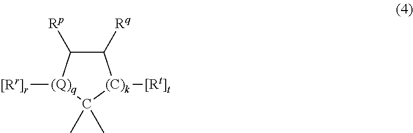

wherein R^{r}, R^{p}, R^{q}, and R^{t} are each independently hydrogen, halogen, oxygen, or C₁₋₁₂ hydrocarbon groups; Q is a direct bond, a carbon, or a divalent oxygen, sulfur, or —N(Z)— where Z is hydrogen, halogen, hydroxy, C₁₋₁₂ alkyl, C₁₋₁₂ alkoxy, or C₁₋₁₂ acyl; r is 0 to 2, t is 1 or 2, q is 0 or 1, and k is 0 to 3, with the proviso that at least two of R^{r}, R^{p}, R^{q}, and R^{t} taken together are a fused cycloaliphatic, aromatic, or heteroaromatic ring. It will be understood that where the fused ring is aromatic, the ring as shown in formula (4) will have an unsaturated carbon-carbon linkage where the ring is fused. When k is one and i is 0, the ring as shown in formula (4) contains 4 carbon atoms, when k is 2, the ring as shown in formula (4) contains 5 carbon atoms, and when k is 3, the ring contains 6 carbon atoms. In an aspect, two adjacent groups (e.g., R^{q} and R^{t} taken together) form an aromatic group, and in another aspect, R^{q} and R^{t} taken together form one aromatic group and R^{r} and R^{p} taken together form a second aromatic group. When R^{q} and R^{t} taken together form an aromatic group, R^{p} can be a double-bonded oxygen atom, i.e., a ketone.

In one aspect, bisphenols (4) can be used in the manufacture of polycarbonates containing phthalimidine carbonate units of formula (4a)

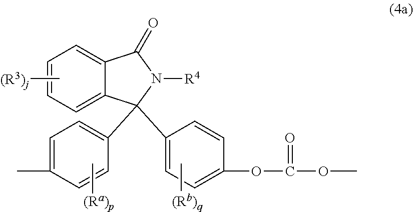

wherein R^{a}, R^{b}, p, and q are as in formula (4), R³ is each independently a C₁₋₆ alkyl group, j is 0 to 4, and R₄ is a C₁₋₆ alkyl, phenyl, or phenyl substituted with up to five C₁₋₆ alkyl groups. In particular, the phthalimidine carbonate units are of formula (4b)

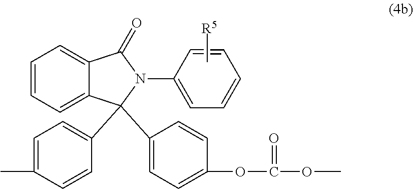

wherein R⁵ is hydrogen or a C₁₋₆ alkyl. In an aspect, R⁵ is hydrogen. Carbonate units (4a) wherein R⁵ is hydrogen can be derived from 2-phenyl-3,3′-bis(4-hydroxy phenyl)phthalimidine (also known as N-phenyl phenolphthalein bisphenol, or “PPPBP”) (also known as 3,3-bis(4-hydroxyphenyl)-2-phenylisoindolin-1-one).

Other bisphenol carbonate repeating units of this type are the isatin carbonate units of formula (4c) and (4d)

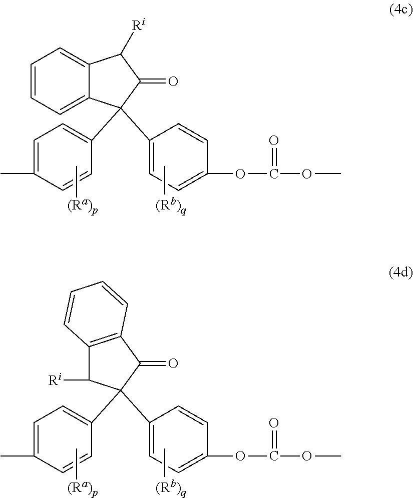

wherein R^{a} and R^{b} are each independently C₁₋₁₂ alkyl, p and q are each independently 0 to 4, and Rⁱ is C₁₋₁₂ alkyl, phenyl, optionally substituted with 1 5 to C₁₋₁₀ alkyl, or benzyl optionally substituted with 1 to 5 C₁₋₁₀ alkyl. In an aspect, R^{a} and R^{b} are each methyl, p and q are each independently 0 or 1, and Rⁱ is C₁₋₄ alkyl or phenyl.

Examples of bisphenol carbonate units derived from bisphenols (4) wherein X^{b} is a substituted or unsubstituted C₃₋₁₈ cycloalkylidene include the cyclohexylidene-bridged, alkyl-substituted bisphenol of formula (4e)

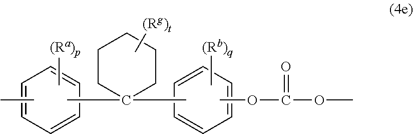

wherein R^{a} and R^{b} are each independently C₁₋₁₂ alkyl, R^{g} is C₁₋₁₂ alkyl, p and q are each independently 0 to 4, and t is 0 to 10. In a specific aspect, at least one of each of R^{a} and R^{b} are disposed meta to the cyclohexylidene bridging group. In another aspect, R^{a} and R^{b} are each independently C₁₋₄ alkyl, R^{g} is C₁₋₄ alkyl, p and q are each 0 or 1, and t is 0 to 5. In another aspect, R^{a}, R^{b}, and R^{g} are each methyl, r and s are each 0 or 1, and t is 0 or 3, specifically 0. For example,

Examples of other bisphenol carbonate units derived from bisphenol (4) wherein X^{b} is a substituted or unsubstituted C₃₋₁₈ cycloalkylidene include adamantyl units (4f) and units (4g)

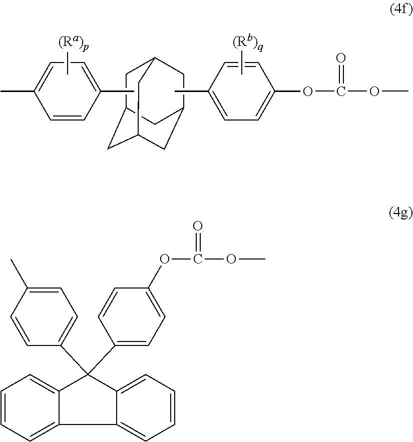

wherein R^{a} and R^{b} are each independently C₁₋₁₂ alkyl, and p and q are each independently 1 to 4. In a specific aspect, at least one of each of R^{a} and R^{b} are disposed meta to the cycloalkylidene bridging group. In an aspect, R^{a} and R^{b} are each independently C₁₋₃ alkyl, and p and q are each 0 or 1. In another specific aspect, R^{a}, R^{b} are each methyl, p and q are each 0 or 1. Carbonates containing units (4a) to (4g) are useful for making polycarbonates with high glass transition temperatures (Tg) and high heat distortion temperatures.

Other useful aromatic dihydroxy compounds of the formula HO—R¹—OH include compounds of formula (5)

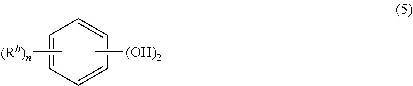

wherein each R^{h} is independently a halogen atom, a C₁₋₁₀ hydrocarbyl such as a C₁₋₁₀ alkyl group, a halogen-substituted C₁₋₁₀ alkyl group, a C₆₋₁₀ aryl group, or a halogen-substituted C₆₋₁₀ aryl group, and n is 0 to 4. The halogen is usually bromine

Some illustrative examples of specific aromatic dihydroxy compounds include the following: 4,4′-dihydroxybiphenyl, 1,6-dihydroxynaphthalene, 2,6-dihydroxynaphthalene, bis(4-hydroxyphenyl)methane, bis(4-hydroxyphenyl)diphenylmethane, bis(4-hydroxyphenyl)-1-naphthylmethane, 1,2-bis(4-hydroxyphenyl)ethane, 1,1-bis(4-hydroxyphenyl)-1-phenylethane, 2-(4-hydroxyphenyl)-2-(3-hydroxyphenyl)propane, bis(4-hydroxyphenyl)phenylmethane, 2,2-bis(4-hydroxy-3-bromophenyl)propane, 1,1-bis (hydroxyphenyl)cyclopentane, 1,1-bis(4-hydroxyphenyl)cyclohexane, 1,1-bis(4-hydroxyphenyl)isobutene, 1,1-bis(4-hydroxyphenyl)cyclododecane, trans-2,3-bis(4-hydroxyphenyl)-2-butene, 2,2-bis(4-hydroxyphenyl)adamantane, alpha, alpha'-bis(4-hydroxyphenyl)toluene, bis(4-hydroxyphenyl)acetonitrile, 2,2-bis(3-methyl-4-hydroxyphenyl)propane, 2,2-bis(3-ethyl-4-hydroxyphenyl)propane, 2,2-bis(3-n-propyl-4-hydroxyphenyl)propane, 2,2-bis(3-isopropyl-4-hydroxyphenyl)propane, 2,2-bis(3-sec-butyl-4-hydroxyphenyl)propane, 2,2-bis(3-t-butyl-4-hydroxyphenyl)propane, 2,2-bis(3-cyclohexyl-4-hydroxyphenyl)propane, 2,2-bis(3-allyl-4-hydroxyphenyl)propane, 2,2-bis(3-methoxy-4-hydroxyphenyl)propane, 2,2-bis(4-hydroxyphenyl)hexafluoropropane, 1,1-dichloro-2,2-bis(4-hydroxyphenyl)ethylene, 1,1-dibromo-2,2-bis(4-hydroxyphenyl)ethylene, 1,1-dichloro-2,2-bis(5-phenoxy-4-hydroxyphenyl)ethylene, 4,4′-dihydroxybenzophenone, 3,3-bis(4-hydroxyphenyl)-2-butanone, 1,6-bis(4-hydroxyphenyl)-1,6-hexanedione, ethylene glycol bis(4-hydroxyphenyl)ether, bis(4-hydroxyphenyl)ether, bis(4-hydroxyphenyl)sulfide, bis(4-hydroxyphenyl)sulfoxide, bis(4-hydroxyphenyl)sulfone, 9,9-bis(4-hydroxyphenyl)fluorine, 2,7-dihydroxypyrene, 6,6′-dihydroxy-3,3,3′,3′-tetramethylspiro(bis)indane (“spirobiindane bisphenol”), 3,3-bis(4-hydroxyphenyl)phthalimide, 2,6-dihydroxydibenzo-p-dioxin, 2,6-dihydroxythianthrene, 2,7-dihydroxyphenoxathin, 2,7-dihydroxy-9,10-dimethylphenazine, 3,6-dihydroxydibenzofuran, 3,6-dihydroxydibenzothiophene, and 2,7-dihydroxycarbazole, resorcinol, substituted resorcinol compounds such as 5-methyl resorcinol, 5-ethyl resorcinol, 5-propyl resorcinol, 5-butyl resorcinol, 5-t-butyl resorcinol, 5-phenyl resorcinol, 5-cumyl resorcinol, 2,4,5,6-tetrafluoro resorcinol, 2,4,5,6-tetrabromo resorcinol, or the like; catechol; hydroquinone; substituted hydroquinones such as 2-methyl hydroquinone, 2-ethyl hydroquinone, 2-propyl hydroquinone, 2-butyl hydroquinone, 2-t-butyl hydroquinone, 2-phenyl hydroquinone, 2-cumyl hydroquinone, 2,3,5,6-tetramethyl hydroquinone, 2,3,5,6-tetra-t-butyl hydroquinone, 2,3,5,6-tetrafluoro hydroquinone, 2,3,5,6-tetrabromo hydroquinone, or the like, or combinations comprising at least one of the foregoing dihydroxy compounds.

Specific examples of bisphenol compounds of formula (3) include 1,1-bis(4-hydroxyphenyl) methane, 1,1-bis(4-hydroxyphenyl) ethane, 2,2-bis(4-hydroxyphenyl) propane (hereinafter “bisphenol A” or “BPA”), 2,2-bis(4-hydroxyphenyl) butane, 2,2-bis(4-hydroxyphenyl) octane, 1,1-bis(4-hydroxyphenyl) propane, 1,1-bis(4-hydroxyphenyl) n-butane, 2,2-bis(4-hydroxy-2-methylphenyl) propane, 1,1-bis(4-hydroxy-t-butylphenyl) propane, 3,3-bis(4-hydroxyphenyl) phthalimidine, 2-phenyl-3,3-bis(4-hydroxyphenyl) phthalimidine (PPPBP), and 1,1-bis(4-hydroxy-3-methylphenyl)cyclohexane (DMBPC). Combinations comprising at least one of the foregoing dihydroxy compounds can also be used. In one specific aspect, the polycarbonate is a linear homopolymer derived from bisphenol A, in which each of A¹ and A² is p-phenylene and Y¹ is isopropylidene in formula (3).

Further to the description above, the term “polycarbonates” is intended to refer to homopolycarbonates (wherein each R¹ in the polymer is the same), copolymers comprising different R¹ moieties in the carbonate (“copolycarbonates”), copolymers comprising carbonate units and other types of polymer units, such as ester units, and combinations comprising at least one of homopolycarbonates and/or copolycarbonates.

A specific type of copolymer is a polyester carbonate, also known as a polyester-polycarbonate. Such copolymers further contain, in addition to recurring carbonate chain units of formula (1), repeating units of formula (6)

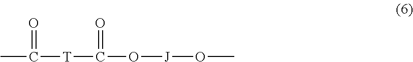

wherein J is a divalent group derived from a dihydroxy compound, and can be, for example, a C₂₋₁₀ alkylene, a C₆₋₂₀ cycloalkylene a C₆₋₂₀ arylene, or a polyoxyalkylene group in which the alkylene groups contain 2 to 6 carbon atoms, specifically 2, 3, or 4 carbon atoms; and T is a divalent group derived from a dicarboxylic acid, and can be, for example, a C₂₋₁₀ alkylene, a C₆₋₂₀ cycloalkylene, or a C₆₋₂₀ arylene. Copolyesters containing a combination of different T and/or J groups can be used. The polyesters can be branched or linear.

In an aspect, J is a C₂₋₃₀ alkylene group having a straight chain, branched chain, or cyclic (including polycyclic) structure. In another aspect, J is derived from an aromatic dihydroxy compound of formula (3) above. In another aspect, J is derived from an aromatic dihydroxy compound of formula (4) above. In another aspect, J is derived from an aromatic dihydroxy compound of formula (5) above.

Aromatic dicarboxylic acids that can be used to prepare the polyester units include isophthalic or terephthalic acid, 1,2-di(p-carboxyphenyl)ethane, 4,4′-dicarboxydiphenyl ether, 4,4′-bisbenzoic acid, or a combination comprising at least one of the foregoing acids. Acids containing fused rings can also be present, such as in 1,4-, 1,5-, or 2,6-naphthalenedicarboxylic acids. Specific dicarboxylic acids include terephthalic acid, isophthalic acid, naphthalene dicarboxylic acid, cyclohexane dicarboxylic acid, or a combination comprising at least one of the foregoing acids. A specific dicarboxylic acid comprises a combination of isophthalic acid and terephthalic acid wherein the weight ratio of isophthalic acid to terephthalic acid is 91:9 to 2:98. In another specific aspect, J is a C₂₋₆ alkylene group and T is p-phenylene, m-phenylene, naphthalene, a divalent cycloaliphatic group, or a combination thereof. This class of polyester includes the poly(alkylene terephthalates).

The molar ratio of ester units to carbonate units in the copolymers can vary broadly, for example 1:99 to 99:1, specifically 10:90 to 90:10, more specifically 25:75 to 75:25, depending on the desired properties of the final composition.

In a specific aspect, the polyester unit of a polyester-polycarbonate is derived from the reaction of a combination of isophthalic and terephthalic diacids (or derivatives thereof) with resorcinol. In another specific aspect, the polyester unit of a polyester-polycarbonate is derived from the reaction of a combination of isophthalic acid and terephthalic acid with bisphenol A. In a specific aspect, the polycarbonate units are derived from bisphenol A. In another specific aspect, the polycarbonate units are derived from resorcinol and bisphenol A in a molar ratio of resorcinol carbonate units to bisphenol A carbonate units of 1:99 to 99:1.

Polycarbonates can be manufactured by processes such as interfacial polymerization and melt polymerization. Branched polycarbonate blocks can be prepared by adding a branching agent during polymerization. These branching agents include polyfunctional organic compounds containing at least three functional groups selected from hydroxyl, carboxyl, carboxylic anhydride, haloformyl, and mixtures of the foregoing functional groups. Specific examples include trimellitic acid, trimellitic anhydride, trimellitic trichloride, tris-p-hydroxy phenyl ethane, isatin-bis-phenol, tris-phenol TC (1,3,5-tris((p-hydroxyphenyl)isopropyl)benzene), tris-phenol PA (4(4(1,1-bis(p-hydroxyphenyl)-ethyl) alpha, alpha-dimethyl benzyl)phenol), 4-chloroformyl phthalic anhydride, trimesic acid, and benzophenone tetracarboxylic acid. The branching agents can be added at a level of 0.05 to 2.0 wt %. Mixtures comprising linear polycarbonates and branched polycarbonates can be used.

A chain stopper (also referred to as a capping agent) can be included during polymerization. The chain stopper limits molecular weight growth rate, and so controls molecular weight in the polycarbonate. chain stoppers include certain mono-phenolic compounds, mono-carboxylic acid chlorides, and/or mono-chloroformates. Mono-phenolic chain stoppers are exemplified by monocyclic phenols such as phenol and C₁-C₂₂ alkyl-substituted phenols such as p-cumyl-phenol, resorcinol monobenzoate, and p-and tertiary-butyl phenol; and monoethers of diphenols, such as p-methoxyphenol. Alkyl-substituted phenols with branched chain alkyl substituents having 8 to 9 carbon atom can be specifically mentioned. Certain mono-phenolic UV absorbers can also be used as a capping agent, for example 4-substituted-2-hydroxybenzophenones and their derivatives, aryl salicylates, monoesters of diphenols such as resorcinol monobenzoate, 2-(2-hydroxyaryl)-benzotriazoles and their derivatives, 2-(2-hydroxyaryl)-1,3,5-triazines and their derivatives, and the like.

Mono-carboxylic acid chlorides can also be used as chain stoppers. These include monocyclic, mono-carboxylic acid chlorides such as benzoyl chloride, C₁-C₂₂ alkyl-substituted benzoyl chloride, toluoyl chloride, halogen-substituted benzoyl chloride, bromobenzoyl chloride, cinnamoyl chloride, 4-nadimidobenzoyl chloride, and combinations thereof; polycyclic, mono-carboxylic acid chlorides such as trimellitic anhydride chloride, and naphthoyl chloride; and combinations of monocyclic and polycyclic mono-carboxylic acid chlorides. Chlorides of aliphatic monocarboxylic acids with less than or equal to 22 carbon atoms are useful. Functionalized chlorides of aliphatic monocarboxylic acids, such as acryloyl chloride and methacryoyl chloride, are also useful. Also useful are mono-chloroformates including monocyclic, mono-chloroformates, such as phenyl chloroformate, alkyl-substituted phenyl chloroformate, p-cumyl phenyl chloroformate, toluene chloroformate, and combinations thereof.

Alternatively, melt processes can be used to make the polycarbonates. The polyester-polycarbonates can also be prepared by interfacial polymerization. Rather than utilizing the dicarboxylic acid or diol per se, the reactive derivatives of the acid or diol, such as the corresponding acid halides, in particular the acid dichlorides and the acid dibromides can be used. Thus, for example instead of using isophthalic acid, terephthalic acid, or a combination comprising at least one of the foregoing acids, isophthaloyl dichloride, terephthaloyl dichloride, or a combination comprising at least one of the foregoing dichlorides can be used.

In addition to the polycarbonates described above, combinations of the polycarbonate with other thermoplastic polymers, for example combinations of homopolycarbonates and/or polycarbonate copolymers with polyesters, can be used. Useful polyesters can include, for example, polyesters having repeating units of formula (6), which include poly(alkylene dicarboxylates), liquid crystalline polyesters, and polyester copolymers. The polyesters described herein are generally completely miscible with the polycarbonates when blended.

The polyesters can be obtained by interfacial polymerization or melt-process condensation as described above, by solution phase condensation, or by transesterification polymerization wherein, for example, a dialkyl ester such as dimethyl terephthalate can be transesterified with ethylene glycol using acid catalysis, to generate poly(ethylene terephthalate). A branched polyester, in which a branching agent, for example, a glycol having three or more hydroxyl groups or a trifunctional or multifunctional carboxylic acid has been incorporated, can be used. Furthermore, it can be desirable to have various concentrations of acid and hydroxyl end groups on the polyester, depending on the ultimate end use of the composition.

Useful polyesters can include aromatic polyesters, poly(alkylene esters) including poly(alkylene arylates), and poly(cycloalkylene diesters). Aromatic polyesters can have a polyester structure according to formula (6), wherein J and T are each aromatic groups as described hereinabove. In an aspect, useful aromatic polyesters can include, for example, poly(isophthalate-terephthalate-resorcinol) esters, poly(isophthalate-terephthalate-bisphenol A) esters, poly[(isophthalate-terephthalate-resorcinol) ester-co-(isophthalate-terephthalate-bisphenol A)] ester, or a combination comprising at least one of these. Also contemplated are aromatic polyesters with a minor amount, e.g., 0.5 to 10 weight percent, based on the total weight of the polyester, of units derived from an aliphatic diacid and/or an aliphatic polyol to make copolyesters. Poly(alkylene arylates) can have a polyester structure according to formula (6), wherein T comprises groups derived from aromatic dicarboxylates, cycloaliphatic dicarboxylic acids, or derivatives thereof. Examples of specifically useful T groups include 1,2-, 1,3-, and 1,4-phenylene; 1,4- and 1,5- naphthylenes; cis- or trans-1,4-cyclohexylene; and the like. Specifically, where T is 1,4-phenylene, the poly(alkylene arylate) is a poly(alkylene terephthalate). In addition, for poly(alkylene arylate), specifically useful alkylene groups J include, for example, ethylene, 1,4-butylene, and bis-(alkylene-disubstituted cyclohexane) including cis- and/or trans-1,4-(cyclohexylene)dimethylene. Examples of poly(alkylene terephthalates) include poly(ethylene terephthalate) (PET), poly(1,4-butylene terephthalate) (PBT), and poly(propylene terephthalate) (PPT). Also useful are poly(alkylene naphthoates), such as poly(ethylene naphthanoate) (PEN), and poly(butylene naphthanoate) (PBN). A specifically useful poly(cycloalkylene diester) is poly(cyclohexanedimethylene terephthalate) (PCT). Combinations comprising at least one of the foregoing polyesters can also be used.

Copolymers comprising alkylene terephthalate repeating ester units with other ester groups can also be useful. Specifically useful ester units can include different alkylene terephthalate units, which can be present in the polymer chain as individual units, or as blocks of poly(alkylene terephthalates). copolymers of this type include poly(cyclohexanedimethylene terephthalate)-co-poly(ethylene terephthalate), abbreviated as PETG where the polymer comprises greater than or equal to 50 mol % of poly(ethylene terephthalate), and abbreviated as PCTG where the polymer comprises greater than 50 mol % of poly(1,4-cyclohexanedimethylene terephthalate).

Poly(cycloalkylene diester)s can also include poly(alkylene cyclohexanedicarboxylate)s. Of these, a specific example is poly(l,4-cyclohexane-dimethano1-1,4-cyclohexanedicarboxylate) (PCCD), having recurring units of formula (7)

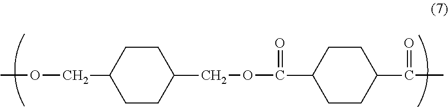

wherein, as described using formula (6), J is a 1,4-cyclohexanedimethylene group derived from 1,4-cyclohexanedimethanol, and T is a cyclohexane ring derived from cyclohexanedicarboxylate or a chemical equivalent thereof, and can comprise the cis-isomer, the trans-isomer, or a combination comprising at least one of the foregoing isomers.

The polycarbonate and polyester can be used in a weight ratio of 1:99 to 99:1, specifically 10:90 to 90:10, and more specifically 30:70 to 70:30, depending on the function and properties desired.

It is desirable for such a polyester and polycarbonate blend to have an MVR of 5 to 150 cc/10 min, specifically 7 to 125 cc/10 min, more specifically 9 to 110 cc/10 min, and still more specifically 10 to 100 cc/10 min, measured at 300° C. and a load of 1.2 kilograms according to ASTM D1238-04.

In another aspect, a polycarbonate can comprise a polysiloxane-polycarbonate copolymer, also referred to as a polysiloxane-polycarbonate. The polydiorganosiloxane (also referred to herein as “polysiloxane”) blocks of the copolymer comprise repeating diorganosiloxane units as in formula (8)

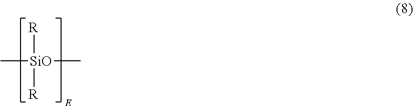

wherein each R is independently a C₁₋₁₃ monovalent organic group. For example, R can be a C₁-C₁₃ alkyl, C₁-C₁₃ alkoxy, C₂-C₁₃ alkenyl group, C₂-C₁₃ alkenyloxy, C₃-C₆ cycloalkyl, C₃-C₆ cycloalkoxy, C₆-C₁₄ aryl, C₆-C₁₀ aryloxy, C₇-C₁₃ arylalkyl, C₇-C₁₃ aralkoxy, C₇⁻C₁₃ alkylaryl, or C₇-C₁₃ alkylaryloxy. The foregoing groups can be fully or partially halogenated with fluorine, chlorine, bromine, or iodine, or a combination thereof. In an aspect, where a transparent polysiloxane-polycarbonate is desired, R is unsubstituted by halogen. Combinations of the foregoing R groups can be used in the same copolymer.

The value of E in formula (8) can vary widely depending on the type and relative amount of each component in the thermoplastic composition, the desired properties of the composition, and like considerations. Generally, E has an average value of 2 to 1,000, specifically 2 to 500, or 2 to 200, more specifically 5 to 100. In an aspect, E has an average value of 10 to 75, and in still another aspect, E has an average value of 40 to 60. Where E is of a lower value, e.g., less than 40, it can be desirable to use a relatively larger amount of the polycarbonate-polysiloxane copolymer. Conversely, where E is of a higher value, e.g., greater than 40, a relatively lower amount of the polycarbonate-polysiloxane copolymer can be used.

A combination of a first and a second (or more) polycarbonate-polysiloxane copolymers can be used, wherein the average value of E of the first copolymer is less than the average value of E of the second copolymer.

In an aspect, the polydiorganosiloxane blocks are of formula (9)

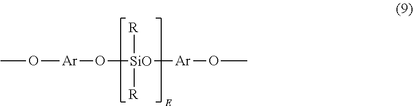

wherein E is as defined above; each R can be the same or different, and is as defined above; and Ar can be the same or different, and is a substituted or unsubstituted C₆-C₃₀ arylene group, wherein the bonds are directly connected to an aromatic moiety. Ar groups in formula (9) can be derived from a C₆-C₃₀ dihydroxyarylene compound, for example a dihydroxyarylene compound of formula (3) or (5) above. dihydroxyarylene compounds are 1,1-bis(4-hydroxyphenyl) methane, 1,1-bis(4-hydroxyphenyl) ethane, 2,2-bis(4-hydroxyphenyl) propane, 2,2-bis(4-hydroxyphenyl) butane, 2,2-bis(4-hydroxyphenyl) octane, 1,1-bis(4-hydroxyphenyl) propane, 1,1-bis(4-hydroxyphenyl) n-butane, 2,2-bis(4-hydroxy-1-methylphenyl) propane, 1,1-bis(4-hydroxyphenyl) cyclohexane, bis(4-hydroxyphenyl sulfide), and 1,1-bis(4-hydroxy-t-butylphenyl) propane. Combinations comprising at least one of the foregoing dihydroxy compounds can also be used.

In another aspect, polydiorganosiloxane blocks are of formula (10)

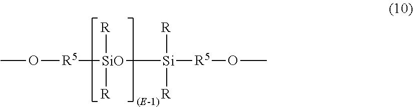

wherein R and E are as described above, and each R⁵ is independently a divalent C₁-C₃₀) organic group, and wherein the polymerized polysiloxane unit is the reaction residue of its corresponding dihydroxy compound. In a specific aspect, the polydiorganosiloxane blocks are of formula (11):

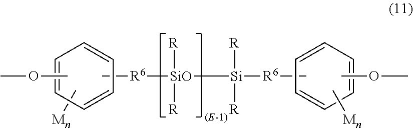

wherein R and E are as defined above. R⁶ in formula (11) is a divalent C₂-C₈ aliphatic group. Each M in formula (11) can be the same or different, and can be a halogen, cyano, nitro, C₁-C₈ alkylthio, C₁-C₈ alkyl, C₁-C₈ alkoxy, C₂-C₈ alkenyl, C₂-C₈ alkenyloxy group, C₃-C₈ cycloalkyl, C₃-C₈ cycloalkoxy, C₆-C₁₀ aryl, C₆-C₁₀ aryloxy, C₇-C₁₂ aralkyl, C₇-C₁₂ aralkoxy, C7-C12 alkylaryl, or C₇-C₁₂ alkylaryloxy, wherein each n is independently 0, 1, 2, 3, or 4.

In an aspect, M is bromo or chloro, an alkyl group such as methyl, ethyl, or propyl, an alkoxy group such as methoxy, ethoxy, or propoxy, or an aryl group such as phenyl, chlorophenyl, or tolyl; R² is a dimethylene, trimethylene or tetramethylene group; and R is a C₁₋₈ alkyl, haloalkyl such as trifluoropropyl, cyanoalkyl, or aryl such as phenyl, chlorophenyl or tolyl. In another aspect, R is methyl, or a combination of methyl and trifluoropropyl, or a combination of methyl and phenyl. In still another aspect, M is methoxy, n is one, R² is a divalent C₁-C₃ aliphatic group, and R is methyl.

Blocks of formula (11) can be derived from the corresponding dihydroxy polydiorganosiloxane (12)

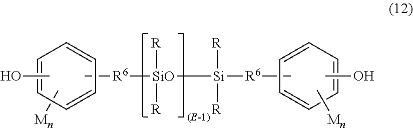

wherein R, E, M, R⁶, and n are as described above. Such dihydroxy polysiloxanes can be made by effecting a platinum-catalyzed addition between a siloxane hydride of formula (13)

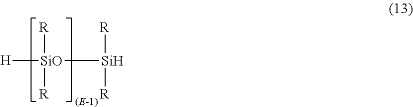

wherein R and E are as previously defined, and an aliphatically unsaturated monohydric phenol. aliphatically unsaturated monohydric phenols include eugenol, 2-alkylphenol, 4-allyl-2-methylphenol, 4-allyl-2-phenylphenol, 4-allyl-2-bromophenol, 4-allyl-2-t-butoxyphenol, 4-phenyl-2-phenylphenol, 2-methyl-4-propylphenol, 2-allyl-4,6-dimethylphenol, 2-allyl-4-bromo-6-methylphenol, 2-allyl-6-methoxy-4-methylphenol and 2-allyl-4,6-dimethylphenol. Combinations comprising at least one of the foregoing can also be used.

The polyorganosiloxane-polycarbonate can comprise 50 to 99 weight percent of carbonate units and 1 to 50 weight percent siloxane units. Within this range, the polyorganosiloxane-polycarbonate copolymer can comprise 70 to 98 weight percent, more specifically 75 to 97 weight percent of carbonate units and 2 to 30 weight percent, more specifically 3 to 25 weight percent siloxane units.

Polyorganosiloxane-polycarbonates can have a weight average molecular weight of 2,000 to 100,000 Daltons, specifically 5,000 to 50,000 Daltons as measured by gel permeation chromatography using a crosslinked styrene-divinyl benzene column, at a sample concentration of 1 milligram per milliliter, and as calibrated with polycarbonate standards.

The polyorganosiloxane-polycarbonate can have a melt volume flow rate, measured at 300° C./1.2 kg, of 1 to 50 cubic centimeters per 10 minutes (cc/10 min), specifically 2 to 30 cc/10 min Mixtures of polyorganosiloxane-polycarbonates of different flow properties can be used to achieve the overall desired flow property.

In another aspect, a polycarbonate material can comprise a flame retardant. In another aspect, a BPA polycarbonate material can comprise a second polycarbonate derived from bisphenol-A, wherein the second polycarbonate is different than the BPA polycarbonate. In another aspect, a BPA polycarbonate material can comprise a second polycarbonate derived from bisphenol-A, wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units. In still another aspect, a BPA polycarbonate can comprise one or more additives selected from at least one of the following: UV stabilizing additives, thermal stabilizing additives, mold release agents, colorants, organic fillers, inorganic fillers, and gamma-stabilizing agents.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As briefly described above, the present disclosure provides a manufacturing process and a promoter catalyst system that can be useful in condensation reactions, such as, for example, the synthesis of bisphenol-A (BPA). BPA can be synthesized by the acid catalyzed condensation of phenol and acetone using either an HCl catalyst or a sulphonated ion exchange resin (IER) catalyst. Due to the inherent low number of acid sites on conventional ion exchange resins, IER processes typically incorporate a promoter system to improve reaction rates. Promoter systems can be bulk, wherein the promoter species is disposed in the reaction medium, or attached, wherein the promoter species is attached to another portion of the catalyst system.

A conventional IER based process utilizes 3-mercaptopropionic acid (3-MPA) as a bulk promoter. While bulk promoters can improve the reaction rate, they require recovery of the promoter species and typically do not provide a high degree of selectivity. For example, in the production of BPA, the use of a 3-MPA promoter can provide a wide range of BPA isomers. Specifically, 3-MPA based systems can result in the production of a significant quantity of o,p-BPA, as opposed to more desirable p,p-BPA. As such, separate isomerization reactions can be necessary to convert o,p-BPA to the more desirable p,p-BPA.

Alternatively, promoter systems can be attached, wherein the promoter is attached to portion of the catalyst system, such as the ion exchange resin. An exemplary attached promoter system utilizes a pyridyl ethylmercapton (PEM) promoter. Conventional attached promoter catalyst systems, such as a PEM based system, can be sensitive to impurities in reactant and recycle streams. For example, in the production of BPA, phenol and acetone reactants can contain impurities such as hydroxyacetone (HA) and methanol, respectively. These impurities can deactivate the catalyst system, resulting in slower reaction rates and shorter catalyst lifetimes.

In one aspect, the present disclosure provides a manufacturing process that can produce high purity BPA, with no or substantially no inorganic, sulfur, or thermally degraded components. In one aspect, the present disclosure provides a manufacturing process that can produce high purity BPA having low or no sulfur present. In another aspect, the present disclosure provides a manufacturing process that does not utilize a bulk promoter, such as, for example, 3-MPA. In another aspect, BPA produced by the methods described herein can exhibit low levels of organic impurities. In yet another aspect, the present disclosure provides a manufacturing process and catalyst system that can provide high purity BPA, suitable for use in food contact polycarbonate applications, healthcare applications, optical applications, or a combination thereof.

In one aspect, the present disclosure provides a promoter catalyst system that is more selective than conventional promoter catalyst systems. In another aspect, the present disclosure provides a manufacturing process and catalyst system for the production of BPA that can selectively produce p,p-BPA without necessitating additional isomerizations reactions. In another aspect, the present disclosure provides a promoter catalyst system that can tolerate impurities, such as hydroxyacetone and methanol, in reactant and/or recycle streams.

In one aspect, the methods described here can be useful for the preparation of BPA. It should also be noted that reactants for bisphenol condensation reactions can comprise phenols, ketones and/or aldehydes, or mixtures thereof. In one aspect, any specific recitation of a ketone, such as acetone, or an aldehyde, is intended to include aspects where only the recited species is used, aspects wherein the other species (e.g., aldehyde for ketone) is used, and aspects wherein a combination of species is used. In other aspects, the methods described herein can be useful for the preparation of other chemical species from, for example, condensation reactions.

In one aspect, phenol reactants can comprise an aromatic hydroxy compound having at least one unsubstituted position, and optionally one or more inert substituents such as hydrocarbyl or halogen at one or more ring positions. In one aspect, an inert substituent is a substituent which does not interfere undesirably with the condensation of the phenol and ketone or aldehyde and which is not, itself, catalytic. In another aspect, phenol reactants are unsubstituted in the position para to the hydroxyl group. As recited here, hydrocarbyl functionalities comprise carbon and hydrogen atoms, such as, for example, alkylene, alkyl, cycloaliphatic, aryl, arylene, alkylarylene, arylalkylene, alkylcycloaliphatic and alkylenecycloaliphatic are hydrocarbyl functions, that is, functions containing carbon and hydrogen atoms.

In one aspect, an alkyl group, if present in a phenol species, comprises from 1 to about 20 carbon atoms, or from 1 to about 5 carbon atoms, or from 1 to about 3 carbon atoms, such as, for example, various methyl, ethyl, propyl, butyl and pentyl isomers. In one aspect, alkyl, aryl, alkaryl and aralkyl substituents are suitable hydrocarbyl substituents on the phenol reactant.

In one aspect, other inert phenol substituents can include, but are not limited to alkoxy, aryloxy or alkaryloxy, wherein alkoxy includes methoxy, ethoxy, propyloxy, butoxy, pentoxy, hexoxy, heptoxy, octyloxy, nonyloxy, decyloxy and polyoxyethylene, as well as higher homologues; aryloxy, phenoxy, biphenoxy, naphthyloxy, etc. and alkaryloxy includes alkyl, alkenyl and alkylnyl-substituted phenolics. Additional inert phenol substituents can include halo, such as bromo, chloro or iodo.

While not intending to be limiting, exemplary phenols can comprise, phenol, 2-cresol, 3-cresol, 4-cresol, 2-chlorophenol, 3-chlorophenol, 4-chlorophenol, 2-tert-butylphenol, 2,4-dimethylphenol, 2-ethyl-6-methylphenol, 2-bromophenol, 2-fluorophenol, 2-phenoxyphenol, 3-methoxyphenol, 2,3,6-trimethylphenol, 2,3,5,6-tetramethylphenol, 2,6-xylenol, 2,6-dichlorophenol, 3,5-diethylphenol, 2-benzylphenol, 2,6-di-tertbutylphenol, 2-phenylphenol, 1-naphthol, 2-naphthol, and/or combinations thereof. In another aspect, phenol reactants can comprise phenol, 2- or 3-cresol, 2,6-dimethylphenol, resorcinol, naphthols, and/or combinations or mixtures thereof. In one aspect, a phenol is unsubstituted.

In one aspect, the phenol starting materials can be commercial grade or better. As readily understood by one of ordinary skill in the art commercial grade reagents may contain measurable levels of typical impurities such as acetone, alpha-methylstyrene, acetophenone, alkyl benzenes, cumene, cresols, water, hydroxyacetone, methyl benzofuran, methyl cyclopentenone, and mesityl oxide, among others.

In one aspect, ketones, if used, can comprise any ketone having a single carbonyl (C═O) group or several carbonyl groups, and which are reactive under the conditions used. In another aspect, ketones can be substituted with substituents that are inert under the conditions used, such as, for example those inert substituents recited above with respect to phenols.

In one aspect, a ketone can comprise aliphatic, aromatic, alicyclic or mixed aromatic-aliphatic ketones, diketones or polyketones, of which acetone, methyl ethyl ketone, diethyl ketone, benzyl, acetyl acetone, methyl isopropyl ketone, methyl isobutyl ketone, acetophenone, ethyl phenyl ketone, cyclohexanone, cyclopentanone, benzophenone, fluorenone, indanone, 3,3,5-trimethylcyclohexanone, anthraquinone, 4-hydroxyacetophenone, acenaphthenequinone, quinone, benzoylacetone and diacetyl are representative examples. In another aspect, a ketone having halo, nitrile or nitro substituents can also be used, for example, 1,3-dichloroacetone or hexafluoroacetone.

Exemplary aliphatic ketones can comprise acetone, ethyl methyl ketone, isobutyl methyl ketone, 1,3-dichloroacetone, hexafluoroacetone, or combinations thereof. In one aspect, the ketone is acetone, which can condense with phenol to produce 2,2-bis-(4-hydroxyphenyl)-propane, commonly known as bisphenol A. In another aspect, a ketone comprises hexafluoroacetone, which can react with two moles of phenol to produce 2,2-bis-(4-hydroxyphenyl)-hexafluoropropane (bisphenol AF). In another aspect, a ketone can comprise a ketone having at least one hydrocarbyl group containing an aryl group, for example, a phenyl, tolyl, naphthyl, xylyl or 4-hydroxyphenyl group.

Other exemplary ketones can include 9-fluorenone, cyclohexanone, 3,3,5-trimethylcyclohexanone, indanone, indenone, anthraquinone, or combinations thereof. Still other exemplary ketones can include benzophenone, acetophenone, 4-hydroxyacetophenone, 4,4′-dihydroxybenzophenone, or combinations thereof.

In one aspect, a ketone reactant can be commercial grade or better. As readily understood by one of ordinary skill in the art commercial grade reagents may contain measurable levels of typical impurities such as aldehydes, acetophenone, benzene, cumene, diacetone alcohol, water, mesityl oxide, and methanol, among others. In one aspect, a ketone, such as, for example, acetone, has less than about 250 ppm of methanol. In another aspect, the inventive catalyst systems of the present invention can tolerate higher concentrations of impurities, such that a ketone can comprise more than 250 ppm of methanol.

In other aspects, the various methods and catalyst systems described herein can be used for the condensation of phenols with aldehydes, for example, with formaldehyde, acetaldehyde, propionaidehyde, butyraldehyde or higher homologues of the formula RCHO, wherein R is alkyl of, for example, 1 to 20 carbon atoms. In one aspect, the condensation of two moles of phenol with one mole of formaldehyde produces bis-(4-hydroxyphenyl)methane, also known as Bisphenol F. It should also be understood that dialdehydes and ketoaldehdyes, for example, glyoxal, phenylglyoxal or pyruvic aldehyde, can optionally be used.

### Promoter Catalyst System—Ion Exchange Resin

The promoter catalyst system of the present disclosure comprises an ion exchange resin catalyst and a promoter. In one aspect, the ion exchange resin can comprise any ion exchange resin suitable for use in the catalyst system of the present invention. In another aspect, the ion exchange resin comprises a cross-linked cationic exchange resin. In another aspect, the ion exchange resin comprises a cross-linked sulfonated ion exchange resin having a plurality of sulfonic acid sites. In yet another aspect, the ion exchange resin is acidic or strongly acidic. In one aspect, at least a portion of the ion exchange resin comprises sodium polystyrene sulfonate. In still other aspects, the ion exchange resin can comprise a monodispersed resin, a polydispersed resin, or a combination thereof.

The specific chemistry of an ion exchange resin or any one or more polymer materials that form a part of an ion exchange resin can vary, and one of skill in the art, in possession of this disclosure, could readily select an appropriate ion exchange resin. In one aspect, the ion exchange resin comprises polystyrene or a derivatized polystyrene. In another aspect, the ion exchange resin comprises a polysiloxane or derivatized polysiloxane. It should also be understood that the catalyst system can, in one aspect, comprise multiple ion exchange resins of the same or varying composition, acidity, and/or degree of cross-linking

In one aspect, the ion exchange resin can be cross-linked with the same or a different polymer material. In various aspects, the degree of cross-linking is from about 1 percent to about 4 percent, for example, about 1, 1.2, 1.4, 1.6, 1.8, 2, 2.2, 2.4, 2.6, 2.8, 3, 3.2, 3.4, 3.6, 3.8, or 4 percent; or from about 1.5 percent to about 2.5 percent, for example, about 1.5, 1.6, 1.7, 1.8, 1.9, 2, 2.1, 2.2, 2.3, 2.4, or 2.5 percent. In other aspects, the degree of cross-linking can be less than 1 percent or greater than 4 percent, and the present invention is not intended to be limited to any particular degree of cross-linking recited here. In a specific aspect, the degree of cross-linking is about 2 percent. In another aspect, the ion exchange resin is not cross-linked While not wishing to be bound by theory, cross-linking of an ion exchange resin is not necessary, but can provide additional stability to the resin and the resulting catalyst system.

In one aspect, the ion exchange resin can be cross-linked using any conventional cross-linking agents, such as, for example, polycyclic aromatic divinyl monomers, divinyl benzene, divinyl toluene, divinyl biphenyl monomers, or combinations thereof.

In other aspects, the ion exchange resin comprises a plurality of acid sites, and has, before modification, at least about 3, at least about 3.5, at least about 4, at least about 5, or more acid milliequivalents per gram (meq/g) when dry. In a specific aspect, the ion exchange resin, before modification, has at least about 3.5 acid milliequivalents per gram when dry. In various aspects, any of the plurality of acid sites on an ion exchange resin can comprise a sulfonic acid functionality, which upon deprotonation produces a sulfonate anion functionality, a phosphonic acid functionality, which upon deprotonation produces a phosphonate anion functionality, or a carboxylic acid functionality, which upon deprotonation produces a carboxylate anion functionality.

Exemplary ion exchange resins can include, but are not limited to, DIAION® SK104, DIAION® SK1B, DIAION® PK208, DIAION® PK212 and DIAION® PK216 (manufactured by Mitsubishi Chemical Industries, Limited), A-121, A-232, and A-131, (manufactured by Rohm & Haas), T-38, T-66 and T-3825 (manufactured by Thermax), LEWATIT® K1131, LEWATIT® K1221 (manufactured by Lanxess), DOWEX® 50W2X, DOWEX® 50W4X, DOWEX® 50W8X resins (manufactured by Dow Chemical), Indion 180, Indion 225 (manufactured by Ion Exchange India Limited), and PUROLITE® CT-222 and PUROLITE® CT-122 (manufactured by Purolite).

### Promoter Catalyst System—Promoter

In one aspect, the promoter of the present invention comprises dimethyl thiazolidine (DMT). In other aspects, the promoter of the present invention can comprise derivatives and/or analogues of dimethyl thiazolidine. In another aspect, the promoter of the present invention can be represented by the formula:

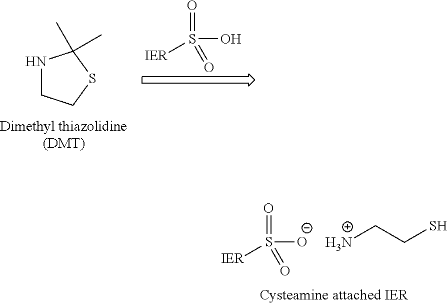

In one aspect, the promoter can be contacted with the ion exchange resin so as to neutralize at least a portion of the available acid sites on the ion exchange resin, and attach thereto. In various aspects, the ion exchange resin is modified by neutralizing from about 18% to about 25% of the available acid sites with the promoter. In another aspect, the promoter is bound to from about 18% to about 25%, for example, about 18, 19, 20, 21, 22, 23, 24, or 25% of the acid sites on the ion exchange resin. In another aspect, the promoter is bound to from about 20% to about 24% of the acid sites on the ion exchange resin. In still another aspect, the promoter is bound to about 22% of the acid sites of the ion exchange resin.

In an exemplary process, the promoter is combined with a solvent to form a mixture. The mixture may further comprise an acid to improve solubility of the promoter. In one aspect, the amount of acid can be sufficient to solubilize the promoter but not enough to impede modification of the ion exchange resin. In one aspect, the amount of acid is typically less than or equal to about 1 equivalent; or less than or equal to about 0.25 equivalents, based on the number of moles of the promoter. Exemplary acids include, but are not limited to, hydrochloric acid (HCl), p-toluenesulfonic acid, trifluorocacetic acid, and acetic acid. In such an aspect, the mixture can be contacted with the ion exchange resin resulting in an ionic linkage between the promoter cation and anion (deprotonated acid site) of the ion exchange resin. Formation of the ionic linkage can thus neutralize the acid site.

The degree of neutralization may be determined in a number of ways. In one aspect, the modified ion exchange resin catalyst can be titrated to determine the amount of remaining acid sites.

Following modification (neutralization), the modified ion exchange resin catalyst can optionally be rinsed with a continuous flow of phenol to remove any remaining amounts of solvent from the modification. Alternatively, if acid was used to improve the solubility of the promoter, the modified ion exchange resin can optionally be rinsed with deionized water prior to rinsing with phenol. In one aspect, removing substantially all of the water is herein defined as removing greater than or equal to about 75%, greater than or equal to about 80%, or greater than or equal to about 85%, based on the total amount of water initially employed.

In one aspect, at least a portion of the promoter is ionically bound to the available acid sites of the ion exchange resin. In another aspect, all or substantially all of the promoter is ionically bound to acid sites of the ion exchange resin. In another aspect, at least a portion of the promoter is covalently bound to at least a portion of the ion exchange resin. In still another aspect, all or substantially all of the promoter is at least covalently bound to the ion exchange resin. In yet another aspect, the degree of attachment or binding between a promoter and an ion exchange resin can vary, such as, for example, covalent binding, ionic binding, and/or other interactions or attraction forces, and the present invention is not intended to be limited to any particular degree of attachment.

### Reactant Impurities

For the manufacture of BPA, both phenol and acetone reactants can contain impurities, such as hydroxyacetone (HA) and methanol, respectively. These reactants can interfere with and/or deactivate catalyst systems, resulting in shortened catalyst lifetimes and/or decreased reaction rates. A conventional approach to prevent such deactivation is to subject the reactants to a pretreatment step, such as an adsorption bed, to remove the impurities.

In one aspect, the DMT attached promoter catalyst system of the present invention can tolerate phenol and alcohol impurities without reducing the lifetime of the catalyst system. In another aspect, the DMT attached promoter catalyst system can tolerate other impurities detrimental to conventional catalyst systems. In yet another aspect, the DMT attached promoter catalyst system can provide performance equivalent to or greater than that of conventional bulk promoter systems. In comparison with a conventional PEM attached promoter catalyst system, the DMT catalyst system can exhibit no significant change in catalyst activity level after exposure to HA. Thus, in one aspect, the DMT catalyst system can eliminate the need for separate purification and/or pretreatment steps.

In one aspect, a manufacturing process using the DMT catalyst system can require a reduced level of pretreatment and/or purification of reactants. In another aspect, a bisphenol manufacturing process can utilize phenol and acetone reactants as received, without the need for a pretreatment step. In still other aspects, the lifetime of a DMT promoter catalyst system, after exposed to HA and/or methanol, can be longer than that for conventional bulk or attached promoter catalyst systems.

In one aspect, the DMT catalyst system can tolerate a greater amount of hydroxyacetone than a comparative PEM catalyst system. In various aspects, upon exposure to about 10 ppm hydroxyacetone, the DMT catalyst system can maintain at least about 60, at least about 65, at least about 70, at least about 75, or at least about 80% of its initial performance after 200 hours of operation, in terms of the amount of p,p-BPA produced. In other aspects, upon exposure to about 10 ppm hydroxyacetone, the DMT catalyst system can maintain at least about 10, at least about 15, at least about 20, or at least about 25% of its initial performance after 500 hours of operation, in terms of the amount of p,p-BPA produced.

As described above, the DMT catalyst system can be more resistant to deactivation than other catalyst systems. In one aspect, the DMT catalyst system can substantially maintain its acid strength after 100 hours of operation under 20 ppm of hydroxyacetone. In various aspects, the acid strength (meq/g) of the DMT catalyst system, after 100 hours of exposure to 20 ppm hydroxyacetone, is within 10%, within 8%, within 6%, within 4%, or within 2% of the acid strength for a DMT catalyst system not exposed to hydroxyacetone. In a specific aspect, the acid strength of the DMT catalyst system, after 100 hours of exposure to 20 ppm hydroxyacetone, is within 5% of the acid strength for a DMT catalyst system not exposed to hydroxyacetone.

In addition to improved resistance to hydroxyacetone, the DMT catalyst system can tolerate exposure to alcohols, such as methanol, with substantially no change in performance. In various aspects, the DMT catalyst system can tolerate up to about 100 ppm, up to about 250 ppm, up to about 500 ppm, up to about 1,000 ppm, up to about 1,500 ppm, up to about 2,000 ppm, up to about 2,500 ppm, up to about 3,000 ppm, up to about 4,000 ppm, up to about 5,000 ppm, up to about 6,000, or more of methanol with no or substantially no detectable decrease in performance. In a specific aspect, the DMT catalyst system can maintain a production rate of p,p-BPA upon exposure to up to about 3,000 ppm methanol. In other aspects, exposure to methanol at each of the concentrations recited above, does not result in any significant change in the selectivity of the DMT catalyst system.

### Recycle Stream Impurities

In addition to reactant impurities, conventional attached promoter systems, such as pyridyl ethylmercaptons (PEM) are also susceptible to impurities in process recycle feeds. In conventional BPA manufacturing processes, a stream of about 10-12% BPA product is recycled to the main reactor, and can be combined with a quantity of fresh acetone. As with reactant impurities, conventional processes can utilize separate purification systems, such as adsorption beds, to remove recycle stream impurities and thus, prevent catalyst deactivation and improve catalyst lifetime.

In one aspect, the DMT attached promoter catalyst system of the present invention can tolerate recycle stream containing 10 to 14 wt % of p,p-BPA, 2 to 4 wt % of o,p-BPA, and 4 to 8 wt % of other BPA impurities, without reducing the lifetime of the catalyst system. In another aspect, the DMT attached promoter catalyst system can tolerate other impurities detrimental to conventional catalyst systems. In yet another aspect, the DMT attached promoter catalyst system can provide performance equivalent to or greater than that of conventional bulk promoter systems. In another aspect, the DMT promoter catalyst system can prevent the need for a separate purification step for process recycle streams.

In one aspect, when using a recycled phenol stream, the DMT catalyst system can provide levels of p,p-BPA that are within about 10%, within about 8%, within about 6%, within about 4%, or within about 2% of values obtained using a fresh phenol stream. In a specific aspect, when using a recycled phenol stream, the DMT catalyst system can provide levels of p,p-BPA that are within about 5% of values obtained using a fresh phenol stream.

Thus, in various aspects, the DMT catalyst system can tolerate recycle stream impurities with no significant degradation in catalyst performance.

### Selectivity

As briefly noted above, the condensation of phenol and acetone to form BPA can yield multiple isomers of BPA, together with other reaction products. For most applications, the p,p-BPA isomer is preferred over the o,p-BPA isomer. In a conventional BPA manufacturing process using a bulk promoter system, isomerization of the BPA reaction product occurs until an equilibrium is reached. The amount of each isomer present at equilibrium depends on the temperature of the reaction medium, as detailed in Table 1, below.

TABLE 1
Equilibrium BPA isomer ratio

| Temperature (° C.) | Equilibrium pp/op ratio |
|---|---|
| 50 | 14.6/1 |
| 60 | 11.6/1 |
| 70 | 10.1.1 |
| 80 | 8.9/1 |
| 90 | 8.1/1 |
| 100 | 6.8/1 |

For conventional bulk promoter systems, higher temperatures can accelerate the reaction rate, but can also accelerate isomerization and the proportion of undesirable o,p-BPA present. Thus, separate isomerization reactors are typically needed to convert produced o,p-BPA to the preferred p,p-BPA isomer. In bulk promoter systems, the isomerization reactor can typically utilize a highly cross-linked (greater than about 8%) ion exchange resin to convert o,p-BPA to p,p-BPA.

Bulk promoter systems typically provide a p,p/o,p-BPA ratio of 10 to 15. In one aspect, the DMT catalyst system can exhibit a higher p,p-BPA to o,p-BPA ratio than a conventional bulk promoter system. In various aspects, the p,p/o,p ratio for the DMT catalyst system can be at least about twice that for conventional bulk promoter systems. In various aspects, a DMT catalyst system can exhibit a p,p/o,p BPA ratio of at least about 20, at least about 30, at least about 40, at least about 50, at least about 60, or more. In another aspect, a DMT catalyst system can exhibit a p,p/o,p-BPA ratio of at least about 25, for example, about 25, 26, 27, 28, 29, 30, 31, 32, 33, 34, 35, 40, or more. In yet another aspect, a DMT catalyst system (22% attachment) can exhibit a p,p/o,p-BPA ratio of from about 25 to about 35.

In another aspect, the improved selectivity of the DMT catalyst system can eliminate the need for a separate isomerization process.

In various aspects, the inventive DMT catalyst system can provide simplified methods for catalyzing condensation reactions. In one aspect, the present invention provides a process for catalyzing a condensation reaction that utilizes a modified ion exchange resin catalyst having an attached dimethyl thiazolidine promoter. In another aspect, the present invention provides a process for catalyzing a condensation reaction that does not utilize a bulk promoter system.

In one aspect, the inventive DMT catalyst system can allow a simplified BPA manufacturing process, wherein one or more of the following are not needed: phenol pretreatment/purification step, acetone pretreatment/purification step, BPA recycle stream purification step, separate isomerization reaction, or a combination thereof. In other aspects, a manufacturing process comprising the inventive DMT catalyst can provide an efficient, selective, longer lifetime catalyst system than conventional attached promoter catalyst systems.

### Properties and Applications of Bpa and Bpa Polycarbonate

In one aspect, BPA synthesized using the methods of the present invention can be useful in producing polycarbonate having enhanced optical properties as compared to a conventional polycarbonate produced from a conventional BPA material. In one aspect, BPA prepared from the methods of the present invention can produce a polycarbonate having good impact strength (ductility). Conventional polycarbonates can age upon exposure to heat, light, and/or over time, resulting in reduced light transmission and color changes within the material. In one aspect, BPA prepared from the methods described herein can exhibit lower levels of inorganic contaminants as compared to conventional BPA materials. In another aspect, BPA prepared from the methods described herein can exhibit lower levels of organic contaminants as compared to conventional BPA materials. In yet another aspect, BPA prepared from the methods described herein can exhibit lower levels of sulfur as compared to conventional BPA materials.

In one aspect, BPA prepared from the methods described herein can have a level of organic impurities of less than about 0.5 wt. %, for example, less than about 0.5 wt. %, less than about 0.4 wt. %, less than about 0.3 wt. %, less than about 0.2 wt. %, or less than about 0.1 wt. %.

Conventional bulk promoter catalyst systems that utilize resin catalyst systems with sulfonic acid groups and 3MPA promotors can leave up to about 20 ppm sulfur or more in the resulting BPA, even after purification. In one aspect, the methods described herein can provide a BPA having less than about 10 ppm, less than about 5 ppm, less than about 4 ppm, less than about 3 ppm, less than about 2 ppm, or less than about 1 ppm sulfur, for example, as measured by combustion and/or coulometric methods. In a specific aspect, the methods described herein can provide a BPA having less than about 2 ppm sulfur. In another aspect, the methods described herein can provide a BPA that is free of or substantially free of sulfur.

In another aspect, the improved purity, for example, reduced sulfur, inorganic contaminants, and/or organic contaminants, of BPA produced using the methods described herein can result in polycarbonate materials having improved color properties. In one aspect, polycarbonate produced from BPA prepared by the methods of the present disclosure can exhibit reduced color, for example, yellowness, as compared to conventional polycarbonate materials, even after aging at elevated temperatures. In one aspect, a polycarbonate produced from BPA prepared by the methods of the present disclosure can exhibit surprisingly low color after aging for 2,000 hours at about 130° C.

In one aspect, the yellowness index (YI), as measured by ASTM D1925, of a 2.5 mm thick polycarbonate plaque formed from a bisphenol-A monomer using the methods of the present disclosure, can be less than about 1.6, for example, less than about 1.6, less than about 1.5, less than about 1.4, or less than about 1.3. In a specific aspect, a 2.5 mm thick polycarbonate plaque can have a yellowness index of less than about 1.5. In another aspect, a 2.5 mm thick polycarbonate plaque can have a yellowness index of less than about 1.3. In another aspect, the yellowness index (YI), as measured by ASTM D1925, of a 2.5 mm thick polycarbonate plaque formed from a bisphenol-A monomer using the methods of the present disclosure, after heat aging for 2,000 hours at about 130 ° C., can be less than about 10, for example, less than about 9, less than about 8, less than about 7, less than about 6, or less than about 5. In a specific aspect, the yellowness index of a 2.5 mm thick polycarbonate plaque, after heat-aging, can be less than about 10. In another aspect, the yellowness index of a 2.5 mm thick polycarbonate plaque, after heat-aging, can be less than about 7.

In another aspect, the yellowness index of a 2.5 mm thick polycarbonate plaque, after heat-aging, can be less than about 5. In another aspect, the yellowness index of a 2.5 mm thick polycarbonate plaque, after heat-aging, can be less than about 2.

In another aspect, BPA polycarbonate produced from the methods described herein can have a purity level suitable for use in optical applications requiring high transmission and low color, wherein the BPA polycarbonate is manufactured from bisphenol-A prepared by contacting at least two chemical reagents with an attached promoter ion exchange resin catalyst system to produce an effluent, and then subjecting the effluent to a solvent crystallization step.

In one aspect, BPA polycarbonate manufactured from bisphenol-A prepared by the methods described herein can have a transmission of at least about 90%, for example, about 90%, 92%, 94%, 96%, 98%, or more, at a thickness of 2.5 mm, as measured by ASTM D1003-00. In other aspects, a BPA polycarbonate, as described herein, can have no or substantially no sulfur impurities. In another aspect, a BPA polycarbonate, as described herein, can have an organic purity of at least about 99.5%. In another aspect, a BPA polycarbonate, as described herein, can have less than or equal to about 150 ppm free hydroxyl groups. In still other aspects, a BPA polycarbonate, as described herein, can have a sulfur concentration of less than about 5 ppm or less than about 2 ppm.

In another aspect, the invention can comprise an article comprising a BPA polycarbonate, for example, a polycarbonate manufactured from BPA produced by the methods described herein. In other aspects, such an article can be selected from at least one of the following: a light guide, a light guide panel, a lens, a cover, a sheet, a bulb, and a film. In a specific aspect, the article can comprise a LED lens. In another aspect, the article can comprise at least one of the following: a portion of a roof, a portion of a greenhouse, and a portion of a veranda.

In other aspects, BPA prepared by the methods described herein can be used to produce polycarbonate resins and/or polycarbonate copolymer materials, for example a polyester-polycarbonate copolymer, a polysiloxane-polycarbonate copolymer, an alkylene terephthalate-polycarbonate copolymer, or a combination thereof. In other aspects, BPA prepared by the methods described herein can be used to produce other polycarbonate copolymers not specifically recited herein, and the present invention is not intended to be limited to any particular polycarbonate and/or polycarbonate copolymer material.

In one aspect, the bisphenol-A, polycarbonate, and article of the present disclosure can comprise any combination of components, purities, and properties described herein, including various aspects wherein any individual component, purity, and/or property, such as, for example, sulfur level, yellowness index, organic purity, and/or transmission can be either included or excluded from the composition. Thus, combinations wherein comprising any one or more components, purities, and/or properties, but excluding other components, purities, and/or properties recited herein are contemplated.

### EXAMPLES OF THE EMBODIMENTS

In one embodiment, a bisphenol-A is prepared by contacting a phenol and at least one of a ketone, an aldehyde, or a combination thereof in the presence of an attached ion exchange resin catalyst comprising a dimethyl thiazolidine promoter, wherein the method does not comprise a pretreatment and/or purification step for the phenol, ketone, and/or aldehydebisphenol.

In the various embodiments, (i) the bisphenol-A has no or substantially no inorganic impurities; and/or (ii) the bisphenol-A has no or substantially no sulfur impurities; and/or (iii) the bisphenol-A has a sulfur concentration of less than about 2 ppm; and/or (iv) the bisphenol A, when formed into a polycarbonate resin and molded into a 2.5 mm plaque, exhibits a yellowness index (YI), as measured by ASTM D1925, of less than about 1.3; and/or (v) the bisphenol-A, when formed into a polycarbonate resin and molded into a 2.5 mm plaque, exhibits a yellowness index (YI), as measured by ASTM D1925, of less than about 10 after heat aging for 2,000 hours at about 130° C.; and/or (vi) the bisphenol-A, when formed into a polycarbonate resin and molded into a 2.5 mm plaque, exhibits a yellowness index (YI), as measured by ASTM D1925, of less than about 7 after heat aging for 2,000 hours at about 130° C.; and/or (vii) the bisphenol-A, when formed into a polycarbonate resin and molded into a 2.5 mm plaque, exhibits a yellowness index (YI), as measured by ASTM D1925, of less than about 2 after heat aging for 2,000 hours at about 130° C.; and/or (viii) the bisphenol-A has a purity level suitable for use in the manufacture of polycarbonate for optical applications and requiring high transmission and low color; and/or (ix) the bisphenol-A has a purity level suitable for the manufacture of food contact grade polycarbonate; and/or the bisphenol A, when formed into a polycarbonate resin, has a transmission level of at least about 90% at a 2.5 mm thickness, as measured by ASTM D1003-00; and/or (x) the bisphenol-A, when formed into a polycarbonate resin, has less than or equal to about 150 ppm free hydroxyl groups; and/or (xii) a polycarbonate or copolymer is prepared from the bisphenol-A of any of the above-described embodiments; and/or (xiii) the polycarbonate or copolymer comprises one or more of a polyester-polycarbonate copolymer, a polysiloxane-polycarbonate copolymer, an alkylene terephthalate-polycarbonate copolymer, or a combination thereof; and/or (xiv) the polycarbonate or copolymer has a yellowness index (YI) of less than about 1.3, as measured by ASTM D1925. when formed into a 2.5 mm thick plaque; and/or (xv) the polycarbonate or copolymer has no or substantially no sulfur impurities; and/or (xvi) the polycarbonate or copolymer has an organic purity of at least about 99.5%; and/or (xvii) the polycarbonate or copolymer has less than or equal to about 150 ppm free hydroxyl groups; and/or (xviii) the polycarbonate or copolymer has a transmission of at least about 90% at 2.5 mm thickness, as measured by ASTM D1003-00; and/or (xix) the polycarbonate or copolymer has a sulfur level of less than about 5 ppm; and/or (xx) the polycarbonate or copolymer has a sulfur level of less than about 2 ppm; and/or (xxi) the polycarbonate or copolymer has a yellowness index (YI) at 2.5 mm thickness, as measured by ASTM D1925, of less than about 1.5; and/or (xxii) the polycarbonate or copolymer has a yellowness index (YI) at 2.5 mm thickness, as measured by ASTM D1925, of less than about 10 after heat aging for 2,000 hours at about 130° C.; and/or (xxiii) the polycarbonate or copolymer has a yellowness index (YI), at 2.5 mm thickness, as measured by ASTM D1925, of less than about 7 after heat aging for 2,000 hours at about 130° C.; and/or (xxiv) the polycarbonate or copolymer has a yellowness index (YI), at 2.5 mm thickness, as measured by ASTM D1925, of less than about 2 after heat aging for 2,000 hours at about 130° C.; and/or (xxv) the polycarbonate or copolymer is an interfacially polymerized polycarbonate; and/or (xxvi) the polycarbonate or copolymer comprises a flame retardant; and/or (xxvii) the polycarbonate or copolymer further comprises a second polycarbonate derived from bisphenol-A, wherein the second polycarbonate is different than the BPA polycarbonate; and/or (xxviii) the second polycarbonate is selected from wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units; and/or (xxix) the polycarbonate or copolymer further comprises one or more additives selected from at least one of the following: UV stabilizing additives, thermal stabilizing additives, mold release agents, colorants, organic fillers, inorganic fillers, and gamma-stabilizing agents; and/or (xxx) an article comprises the bisphenol-A and/or the polycarbonate or copolymer of any of the above-described embodiments; and/or (xxxi) the article is selected from at least one of the following: a light guide, a light guide panel, a lens, a cover, a sheet, a bulb, and a film; and/or (xxxii) the article is a LED lens; and/or (xxxiii) the article comprises at least one of the following: a portion of a roof, a portion of a greenhouse, and a portion of a veranda.

### Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Hydroxyacetone Tolerance

In a first example, a single column reactor was utilized to determine the inventive catalyst system's tolerance for hydroxyacetone (HA) impurities. Parallel reactions were performed: one with 20 ppm HA present in the phenol reactant, the other without HA in the phenol reactant. Reactions were carried out at 75° C., for 100 hours, using 7.5 wt.% acetone, and at WHSV of 20. The ion exchange resin utilized was Lanxess K1221 SH, modified to a level of 20% with the inventive DMT promoter.

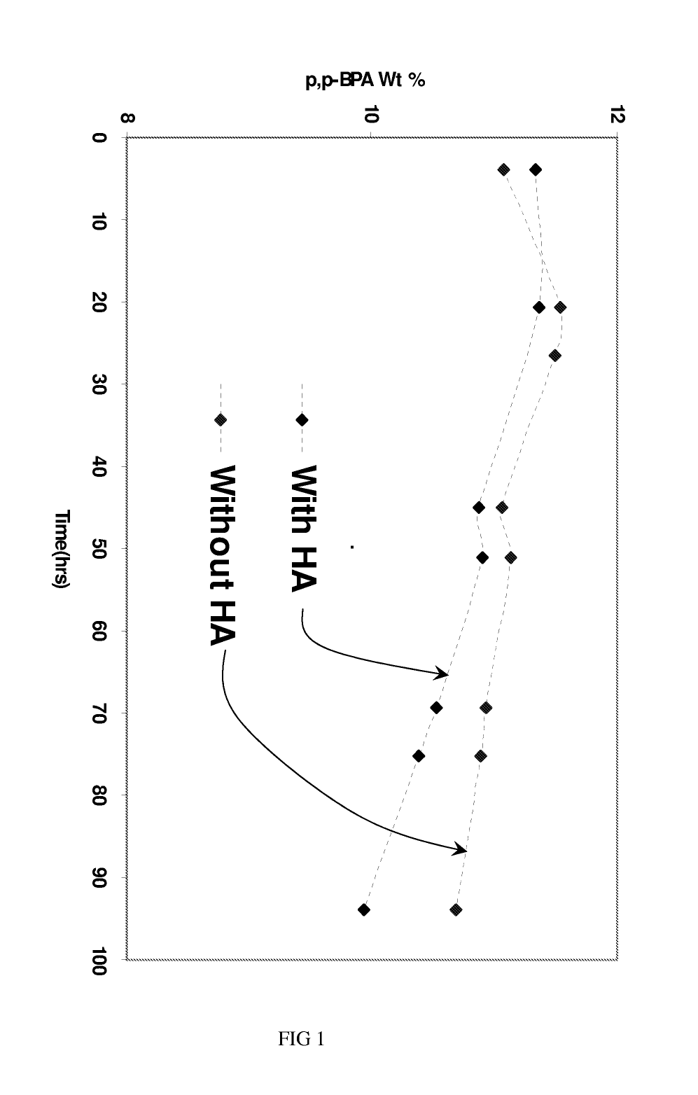

The amount of p,p-BPA produced was then monitored over time. As illustrated in FIG. 1, the reaction occurring in the presence of HA exhibited nearly identical performance to the reaction without HA. After 94 hours, the amount of acetone converted to p,p-BPA was 41% in the reaction without HA, and 38% in the reaction with HA.

The reduction in acid strength (meq/g) of the catalyst system after the 100 hour test was 11.04% for the reaction without HA vs. 15.41% for the reaction with HA present. Thus, only a 4.37% difference in catalyst acid strength was observed between the HA and HA free reactions after 100 hours of operation.

### 2. Methanol Spiking

In a second example, BPA synthesis experiments were performed, wherein the acetone reactant was spiked with methanol. In a first spiking experiment, a single column reactor was operated (WHSV=2, 65° C.) in continuous fashion with an acetone concentration of about 5%. The amount of p,p-BPA formed was monitored over time, as the column feed was periodically spiked with various levels of methanol.

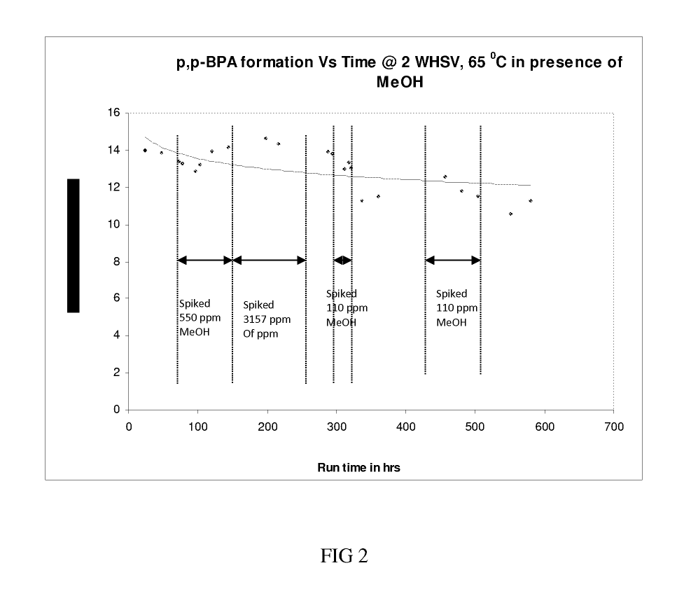

FIG. 2 illustrates the amount of p,p-BPA produced as the column was spiked with methanol (550 ppm, 3157 ppm, and 110 ppm). The observed deactivation profile was identical to that expected when no methanol is present. Thus, the presence of methanol has no detectable effect on the performance of the catalyst system and the formation of p,p-BPA.

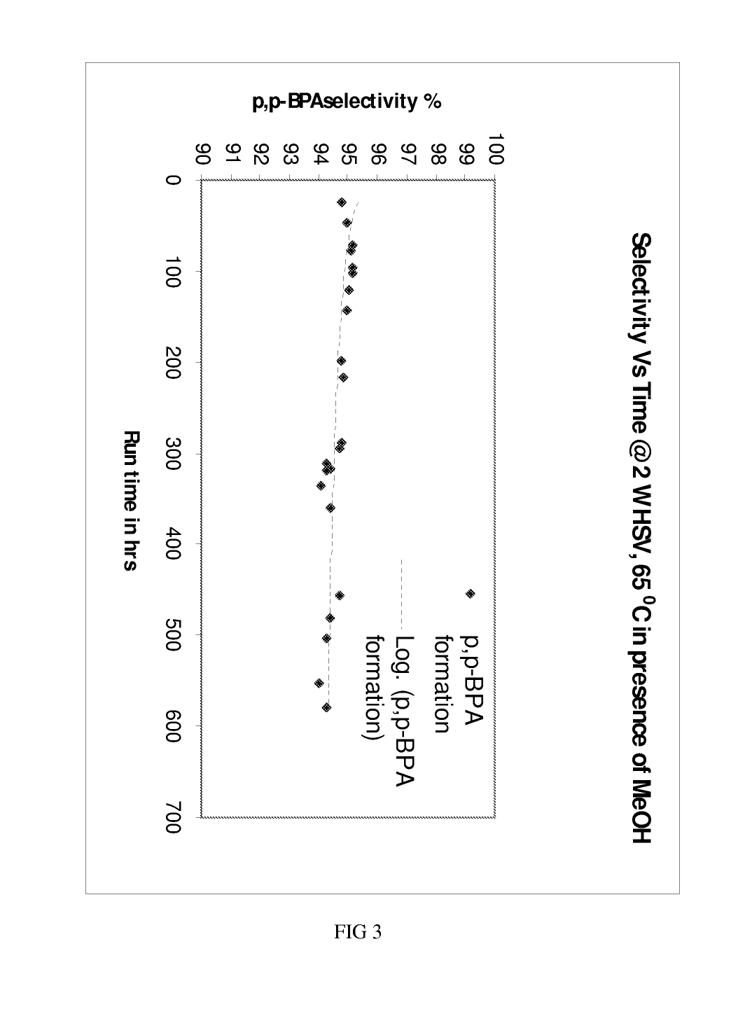

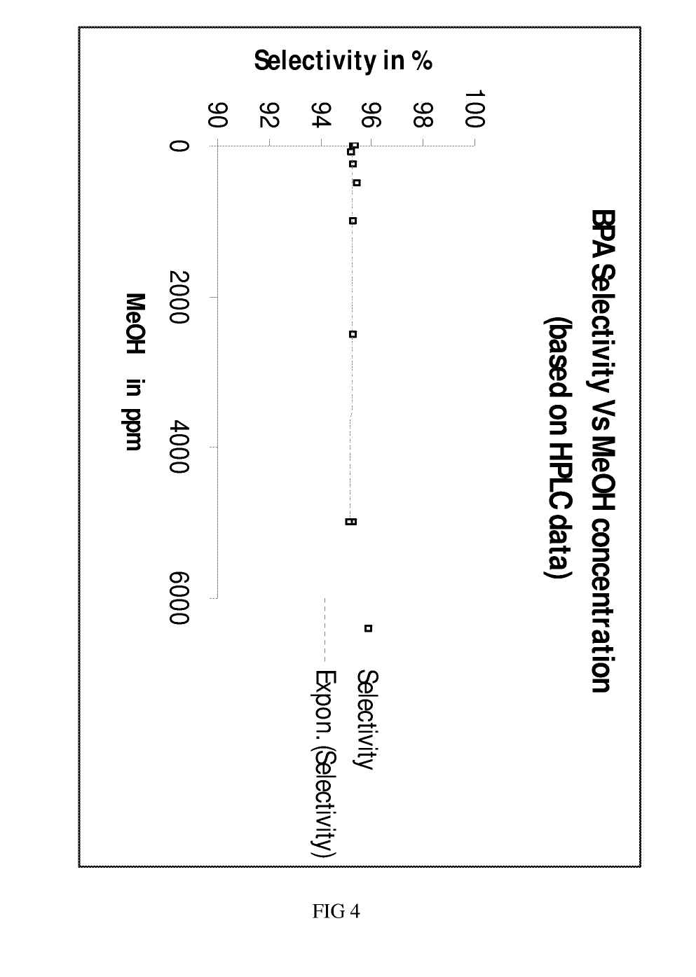

Similarly, FIG. 3 illustrates the selectivity of the inventive catalyst system in the same methanol spiking experiment illustrated in FIG. 2. The presence of methanol in the reaction did not have an effect on the high selectivity of the DMT catalyst towards p,p-BPA. In a separate batch reaction using 5.59% acetone (4 hours at 65° C.), the amount of methanol present in the system was varied between 0 and 5,000 ppm. The selectivity was then monitored as the concentration of methanol in the system varied. As illustrated in FIG. 4, the inventive DMT catalyst system exhibited virtually no change in selectivity over the varying concentration range of methanol.

In yet another set of batch reactions (5.59 wt. % acetone, 4 hours at 65° C.), one reaction was conducted with no methanol present, whereas the second reaction had 1.27 ml of methanol added to the reactants. The concentration of specific reaction products was then determined The amount of o,p-BPA produced with no methanol present was 0.279%, compared to 0.298% when methanol was added. Similarly, the amount of p,p-BPA produced with no methanol present was 9.935%, compared to 10.667% when methanol was added. Thus, the addition of methanol with the DMT catalyst system had no adverse effect on the production of p,p-BPA at 65 C.

In another batch reaction conducted at 85° C. (5.59 wt. % acetone, 30 hours), a series of individual reactions were performed at varying methanol concentrations ranging from 0 to 8.94 wt. %. The amount of p,p-BPA produced over time was measured for each reaction, and is illustrated in FIG. 5. Thus, the inventive DMT catalyst system can remain unaffected by up to at least about 8.9% methanol.

### 3. Isomerization OF o,p-BPA in Attached Promoter System

In a third example, a single column reactor was operated (WHSV 1 and 2) at 65° C. and 75° C. with a reactant feed of 4.5 wt. % acetone and phenol with 2% o,p-BPA. The catalyst system comprised a 2% cross-linked Al21 ion exchange resin with 22% attached dimethyl thiazolidine (DMT).

As detailed in Table 2, below, the DMT catalyst provides effective isomerization and selectivity for the production of p,p-BPA. The DMT catalyst provided a high ratio of p,p-BPA/o,p-BPA and a high degree of selectivity. It should also be noted that isomerization to p,p-BPA increases with increasing o,p-BPA content in the reactor, indicating the usefulness of the inventive catalyst system for acting as a stand-alone catalyst, without the need for a separate isomerization reactor.

TABLE 2
Isomerization Experiment Data

| Temp, ° C. | 65 | 75 | 65 | 75 | 65 | 75 |
|---|---|---|---|---|---|---|
| WHSV | 1.00 | 1.00 | 2.00 | 2.00 | 2.00 | 2.00 |
| % o,p-BPA | 1.00 | 1.00 | 1.00 | 1.00 | 2.00 | 2.00 |
| p,p/o,p-BPA | 28.67 | 23.78 | 32.16 | 27.87 | 64.23 | 42.69 |
| (diff) | | | | | | |
| Selectivity | 95.20 | 94.40 | 95.57 | 94.98 | 96.67 | 95.78 |

### 4. Preparation of Bpa and Polycarbonate

In another example, BPA samples from different sources (e.g., BPA process catalysts and promotors) were used to produce polycarbonate resins. The polycarbonate resins were produced in a single production facility using an interfacial polymerization process. Molded plaques were then prepared from polycarbonate resin stabilized with 0.05 wt. % IRGAFOS® 168 trisarylphosphite processing stabilizer.

The sulfur content and organic purity of each BPA sample were determined. Sulfur measurements were performed using combustion and coulometric method for total sulfur determination. Organic purity was determined using ultraviolet detection after high performance liquid chromatography separation (see HPLC method in Nowakowska et al., Polish J. Appl. Chem., X1(3), 247-254, 1996). The organic purity is defined as 100 wt. % less the sum of known and unknown impurities detected via ultraviolet radiation at 280nm.

The color of each 2.5 mm polycarbonate plaque was determined after molding (YID, as well as after heat aging for 2,000 hours at 130° ^{C.} (YI,2000hrs 130C), according to ASTM D1925, Table 3, below illustrates the color, purity, and sulfur concentration for each sample. Samples prepared using BPA from a conventional bulk promoter system, wherein an ion exchange resin with sulfonic acid groups is used in combination with a 3MPA promoter, as identified as “BP” in the BPA process column. Samples prepared prepared using BPA from a production process using hydrochloric acid as a catalyst are identified as “HCl” in the BPA process column. Samples prepared using BPA from the inventive attached promoter methods described herein are identified as “AP” in the BPA process column.

TABLE 3
Color and Purity Analysis of BPA Materials.

| | | YI | | | BPA process |
|---|---|---|---|---|---|
| | YI | (2000 hrs | BPA purity | Sulfur | catalyst/ |
| Example | (—) | 130 C.) | (% w) | (ppm) | promoter |
| Comp. Ex. 1 | 1.88 | 13.40 | 99.44 | 25 | BP |
| Comp. Ex. 2 | 1.85 | 13.07 | 99.52 | 23 | BP |
| Comp. Ex. 3 | 1.96 | 13.37 | 99.45 | 25 | BP |
| Comp. Ex. 4 | 1.78 | 13.20 | 99.52 | 23 | BP |
| Comp. Ex. 5 | 2.01 | 13.61 | 99.44 | 25 | BP |
| Comp. Ex. 6 | 1.59 | 10.29 | 99.54 | 19 | BP |
| Comp. Ex. 7 | 1.65 | 11.74 | 99.47 | 17 | BP |
| Comp. Ex. 8 | 1.47 | 10.92 | 99.45 | 21 | BP |
| Comp. Ex. 9 | 1.80 | 10.61 | 99.39 | 23 | BP |
| Comp. Ex. 10 | 1.57 | 14.33 | 99.50 | 18 | BP |
| Comp. Ex. 11 | 1.49 | 12.40 | 99.51 | 16 | BP |
| Comp. Ex. 12 | 1.39 | 10.01 | 99.57 | 18 | BP |
| Comp. Ex. 13 | 1.65 | 11.72 | 99.47 | 21 | BP |
| Comp. Ex. 14 | 1.69 | 10.76 | 99.61 | <2 | HCl |
| Comp. Ex. 15 | 1.66 | 10.45 | 99.62 | <2 | HCl |
| Ex. 16 | 1.20 | 6.79 | 99.53 | <2 | AP |
| Ex. 17 | 1.35 | 6.24 | 99.54 | <2 | AP |
| Ex. 18 | 1.26 | 6.72 | 99.54 | <2 | AP |
| Ex. 19 | 1.29 | 7.63 | 99.57 | <2 | AP |
| Ex. 20 | 1.27 | 8.66 | 99.50 | <2 | AP |
| Ex. 21 | 1.31 | 8.71 | 99.56 | <2 | AP |
| Ex. 22 | 1.25 | 4.93 | 99.78 | <2 | AP |
| Ex. 23 | 1.39 | 8.92 | 99.55 | <2 | AP |
| Ex. 24 | 1.42 | 8.04 | 99.57 | <2 | AP |
| Ex. 25 | 1.33 | 5.38 | 99.75 | <2 | AP |
| Ex. 26 | 1.36 | 4.57 | 99.78 | <2 | AP |

The BPA prepared using conventional bulk promoter systems has about 20 ppm sulfur, even after purification of the monomer. The BPA prepared using HCl exhibited a sulfur level of less than about 2 ppm. Similarly, the BPA prepared from the attached prompter systems described herein exhibited less than about 2 ppm sulfur (i.e., a level below the detection limit of the measurement equipment).

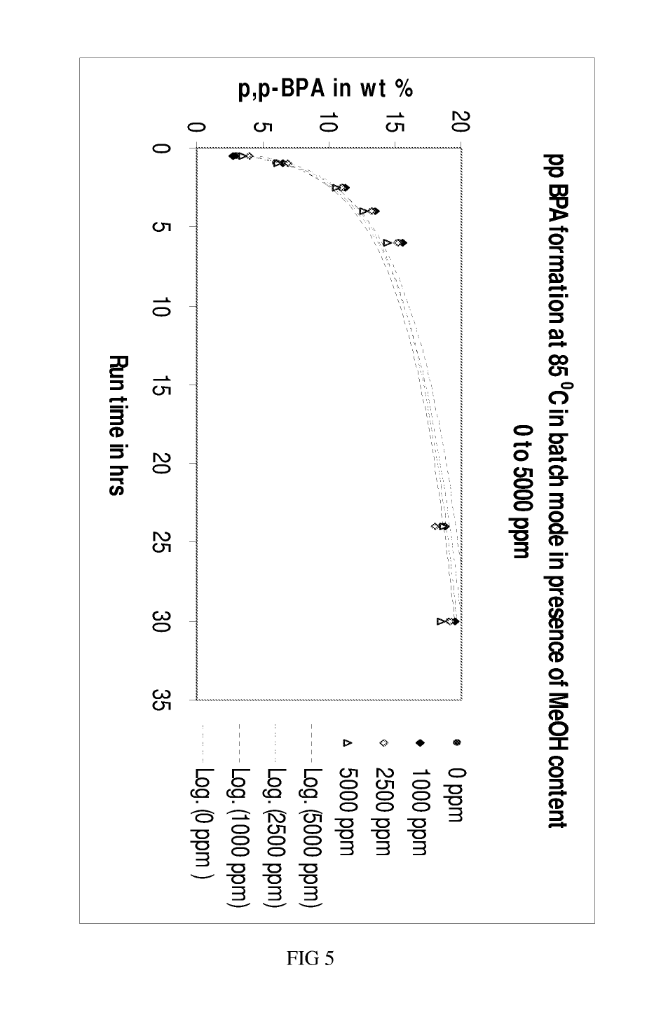

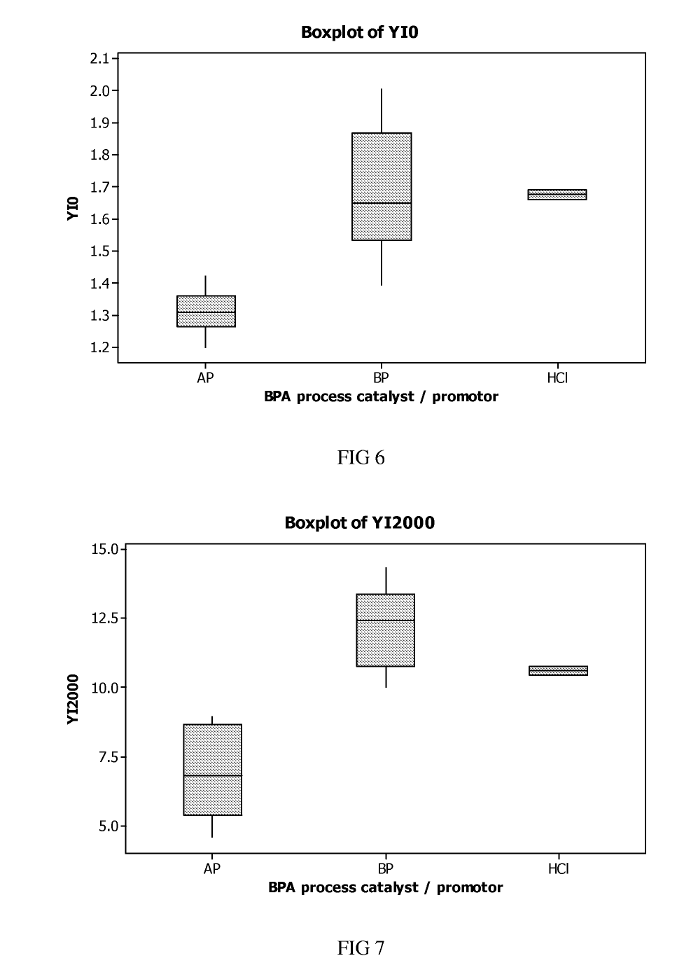

As detailed in Table 3, the color (i.e., yellowing) of plaques prepared from polycarbonate from each of the BPA samples was measured. Polycarbonate resins prepared from conventional bulk promoter (BP) and HCl derived BPA exhibited substantially higher yellowing than resins prepared from attached promoter (AP) derived BPA, both for as-molded plaques and heat-aged plaques. Graphical summaries of the color measurements (yellowness) after molding and after heat aging for 2,000 hours at 130° C. are illustrated in FIGS. 6 and 7, respectively. For both the as-molded and heat-aged plaques, polycarbonate resins produced from BPA prepared by the attached promoter methods of the present disclosure exhibited significantly less yellowing, as compared to polycarbonate resins produced from HCl and conventional bulk promoter (BP) BPA.

While BPA prepared from HCl can exhibit good purity and low sulfur levels, it does not provide the reduced yellowing benefit obtained for BPA prepared with the attached promoter methods described in the present disclosure. BPA prepared from conventional bulk promoter (BP) systems exhibits both higher sulfur content and yellowing, as compared to BPA prepared with the attached promoter methods of the present disclosure.

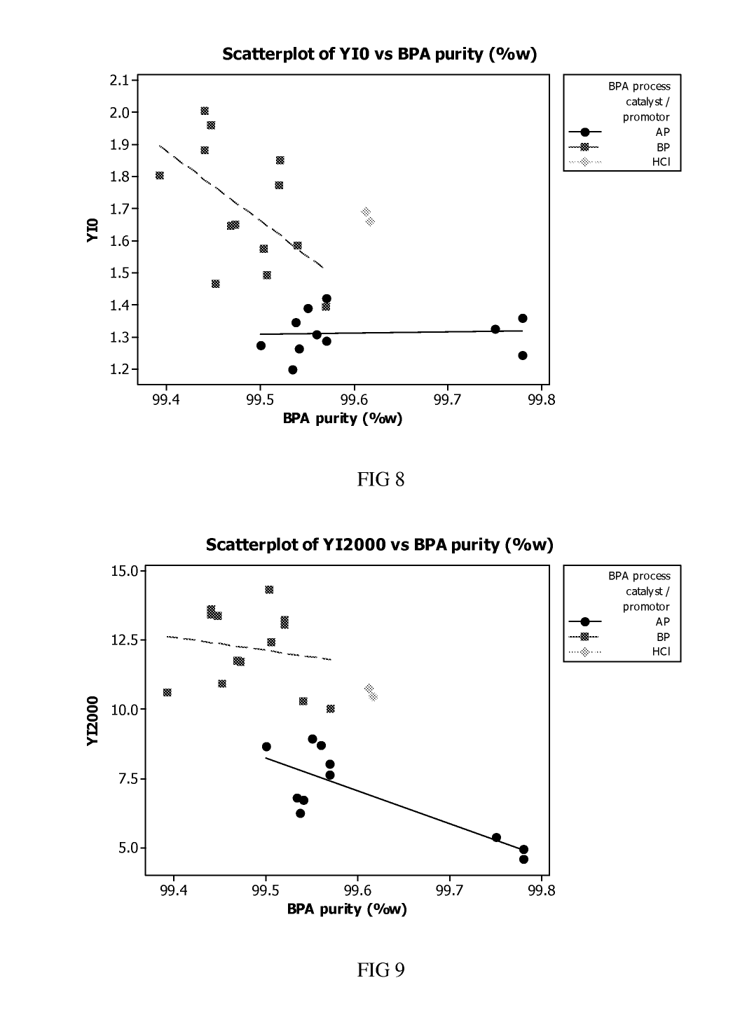

Plots of BPA purity versus color (i.e., yellowing) for as-molded plaques and for heat-aged plaques, are illustrated in FIGS. 8 and 9.

Statistical analysis (ANOVA) indicates a significant difference (95% confidence) between the AP derived samples and the other materials for both starting color as well as color after heat aging. Comparing inventive examples 16-26 with comparative examples 14 and 15 shows that this improved color is not just related to the sulfur content in the resin, which is one of the differences when comparing AP and BP derived materials. The overall organic purity itself is not the only factor in determining color and color stability either as shown in the more detailed graphs (FIGS. 3 & 4) below. Although a higher organic monomer purity appears to lead to lower yellowing for the BP derived samples, the AP derived samples clearly outperform the BP materials at a given purity of e.g. 99.55%.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A bisphenol-A prepared by contacting a phenol and at least one of a ketone, an aldehyde, or a combination thereof in the presence of an attached ion exchange resin catalyst comprising a dimethyl thiazolidine promoter, wherein the method does not comprise a pretreatment and/or purification step for the phenol, ketone, and/or aldehyde.

2. The bisphenol-A of claim 1, having no or substantially no inorganic impurities.

3. The bisphenol-A of claim 1, having no or substantially no sulfur impurities.

4. The bisphenol-A of claim 1, having a sulfur concentration of less than about 2 ppm.

5. The bisphenol-A of claim 1, wherein when formed into a polycarbonate resin and molded into a 2.5 mm plaque, exhibits a yellowness index (YI), as measured by ASTM D1925, of less than about 1.3.

6. The bisphenol-A of claim 1, wherein when formed into a polycarbonate resin and molded into a 2.5 mm plaque, exhibits a yellowness index (YI), as measured by ASTM D1925, of less than about 10 after heat aging for 2,000 hours at about 130° C.

7. The bisphenol-A of claim 6, wherein when formed into a polycarbonate resin and molded into a 2.5 mm plaque, exhibits a yellowness index (YI), as measured by ASTM D1925, of less than about 7 after heat aging for 2,000 hours at about 130° C.

8. The bisphenol-A of claim 7, wherein when formed into a polycarbonate resin and molded into a 2.5 mm plaque, exhibits a yellowness index (YI), as measured by ASTM D1925, of less than about 2 after heat aging for 2,000 hours at about 130° C.

9. The bisphenol-A of claim 1, having a purity level suitable for use in the manufacture of polycarbonate for optical applications and requiring high transmission and low color.

10. The bisphenol-A of claim 1, having a purity level suitable for the manufacture of food contact grade polycarbonate.

11. The bisphenol-A of claim 1, wherein when formed into a polycarbonate resin, has a transmission level of at least about 90% at a 2.5 mm thickness, as measured by ASTM D1003-00.

12. The bisphenol-A of claim 1, wherein when formed into a polycarbonate resin, has less than or equal to about 150 ppm free hydroxyl groups.

13. A polycarbonate or copolymer prepared from the bisphenol-A of claim 1.

14. The polycarbonate or copolymer of claim 13, comprising one or more of a polyester-polycarbonate copolymer, a polysiloxane-polycarbonate copolymer, an alkylene terephthalate -polycarbonate copolymer, or a combination thereof.

15. The polycarbonate or copolymer of claim 13, having a yellowness index (YI) of less than about 1.3, as measured by ASTM D1925, when formed into a 2.5 mm thick plaque.

16. The polycarbonate or copolymer of claim 13, having no or substantially no sulfur impurities.

17. The polycarbonate or copolymer of claim 13, having an organic purity of at least about 99.5%.

18. The polycarbonate or copolymer of claim 13, having less than or equal to about 150 ppm free hydroxyl groups.

19. The polycarbonate or copolymer of claim 13, having a transmission of at least about 90% at 2.5 mm thickness, as measured by ASTM D1003-00.

20. The polycarbonate or copolymer of claim 13, having a sulfur level of less than about 5 ppm.

21. The polycarbonate or copolymer of claim 20, having a sulfur level of less than about 2 ppm,

22. The polycarbonate or copolymer of claim 13, having a yellowness index (YI) at 2.5 mm thickness, as measured by ASTM D1925, of less than about 1.5.

23. The polycarbonate or copolymer of claim 13, having a yellowness index (YI) at 2.5 mm thickness, as measured by ASTM D1925, of less than about 10 after heat aging for 2,000 hours at about 130° C.

24. The polycarbonate or copolymer of claim 23, having a yellowness index (YI), at 2.5 mm thickness, as measured by ASTM D1925, of less than about 7 after heat aging for 2,000 hours at about 130° C.

25. The polycarbonate or copolymer of claim 24, having a yellowness index (YI), at 2.5 mm thickness, as measured by ASTM D1925, of less than about 2 after heat aging for 2,000 hours at about 130° C.

26. The polycarbonate or copolymer of claim 13, wherein the polycarbonate is an interfacially polymerized polycarbonate.

27. The polycarbonate or copolymer of claim 13, comprising a flame retardant.

28. The polycarbonate or copolymer of claim 13 any of claims 13-27, further comprising a second polycarbonate derived from bisphenol-A, wherein the second polycarbonate is different than the BPA polycarbonate.

29. The polycarbonate or copolymer of claim 28, wherein the second polycarbonate is selected from wherein the second polycarbonate is selected from at least one of the following: a homopolycarbonate derived from a bisphenol; a copolycarbonate derived from more than on bisphenol; and a copolymer derived from one or more bisphenols and comprising one or more aliphatic ester units or aromatic ester units or siloxane units.

30. The polycarbonate or copolymer of claim 13, further comprising one or more additives selected from at least one of the following: UV stabilizing additives, thermal stabilizing additives, mold release agents, colorants, organic fillers, inorganic fillers, and gamma-stabilizing agents.

31. An article comprising the bisphenol-A of claim 1 and/or the polycarbonate or copolymer of claim 13.

32. The article of claim 31, wherein the article is selected from at least one of the following: a light guide, a light guide panel, a lens, a cover, a sheet, a bulb, and a film.

33. The article of claim 31, wherein the article is a LED lens.

34. The article of claim 31, wherein the article comprises at least one of the following: a portion of a roof, a portion of a greenhouse, and a portion of a veranda.
